(19) United States
(12) Patent Application Publication
(10) Pub. No.: US 2026/0049064 A1
(43) Pub. Date: Feb. 19, 2026

(54) PROTEIN:PROTEIN INTERACTION INHIBITORS

(71) Applicants: Purdue Research Foundation, West Lafayette, IN (US); University of Pittsburgh - Of the Commonwealth System of Higher Education, Pittsburgh, PA (US)

(72) Inventors: Chang-Deng Hu, West Lafayette, IN (US); Andrew M. Asberry, Indianapolis, IN (US); Xinpei Cai, San Diego, CA (US); Xuehong Deng, West Lafayette, IN (US); Hunter Sims, Decatur, GA (US); Carlos J. Camacho, Pittsburgh, PA (US); Mingji Dai, Alpharetta, GA (US)

(73) Assignees: Purdue Research Foundation, West Lafayette, IN (US); University of Pittsburgh - Of the Commonwealth System of Higher Education, Pittsburgh, PA (US)

(21) Appl. No.: 19/104,961

(22) PCT Filed: Aug. 18, 2023
(86) PCT No.: PCT/US2023/030604

§ 371 Date: Feb. 19, 2025

Related U.S. Application Data
(60) Provisional application No. 63/399,485, filed on Aug. 19, 2022.

(51) Int. Cl. C07D 261/08; A61K 31/42; A61K 31/4709; C07D 413/12

(52) U.S. Cl. CPC C07D 261/08; A61K 31/42; A61K 31/4709; C07D 413/12

(57) ABSTRACT

Disclosed are inhibitors of a protein-protein interaction between protein arginine methyltransferase 5 (PRMT5) and methylosome protein 50 (MEP50) based on isoxazolyl methoxyphenyl derivatives. Further disclosed are pharmaceutical compositions comprising PRMT5:MEP50 inhibitors and methods of inhibiting protein arginine methyltransferase 5 (PRMT5) using PRMT5.MEP50 inhibitors or pharmaceutical compositions comprising PRMT5:MEP50 inhibitors.

### CROSS-REFERENCE TO RELATED APPLICATIONS

This application claims priority to U.S. provisional patent application No. 63/399,485, filed Aug. 19, 2022.

### STATEMENT OF GOVERNMENT SUPPORT

This invention was made with government support under W81XWH-16-1-0394 awarded by the U.S. Army and under CA212403 and GM128570 awarded by the National Institutes of Health. The government has certain rights in the invention.

### TECHNICAL FIELD

The disclosure herein pertains to inhibitors of a protein-protein interaction between protein arginine methyltransferase 5 (PRMT5) and methylosome protein 50 (MEP50). Synthesis of PRMT5:MEP50 inhibitors, compositions containing PRMT5:MEP50 inhibitors, and therapeutic uses of PRMT5:MEP50 inhibitors are also disclosed.

### INTRODUCTION

Protein arginine methyltransferase 5 (PRMT5) is one of nine members of the PRMT family of methyltransferases and is responsible for the majority of symmetric dimethylation of arginine residues in cells. Through post-translational modification of signaling proteins such as p53, E2F and EGFR as well as epigenetic regulation of target gene expression via symmetric dimethylation of histones (H4R3, H3R2, H3R8 and H2AR3), PRMT5 is required for many cellular processes including cell proliferation, differentiation, survival, DNA damage response, and RNA splicing. PRMT5 is significantly dysregulated or overexpressed in multiple cancers, and its overexpression appears to correlate with cancer progression and poor clinical outcomes. One of the major mechanisms to account for its putative oncogenic role is epigenetic repression of tumor suppressors such as Rb1, ST7, PTEN, and p53. Structural studies have demonstrated that PRMT5 forms a complex with methylosome protein 50 (MEP50) for biological enzymatic function as well as formation of higher order complexes. Based on in vitro biochemical assays, the presence of MEP50 increases the enzymatic activity of PRMT5 by 100-fold, suggesting that MEP50 is an obligate cofactor. In addition, multiple PRMT5 interacting proteins appear to serve as adaptors to specifically recruit various substrates or dictate biological activity.

It has recently been demonstrated that PRMT5 can also promote prostate cancer cell growth and confer therapy resistance through transcriptional activation of the androgen receptor (AR) in both hormone naïve prostate cancer (HNPC) and castration resistant prostate cancer (CRPC) through interaction with cofactor pICln. In addition to mediating resistance to AR targeting, it has been demonstrated that PRMT5 also mediates activation of DNA damage response pathway in response to fractionating ionizing radiation, providing two distinct mechanisms for PRMT5 to mediate therapy resistance for prostate cancer cells at two separate stages of disease. These findings suggest that targeting PRMT5 and its interacting proteins including substrate adaptors such as pICln, COPR5 and RIOK1 may offer a unique and potentially specific approach to target PRMT5 in a context specific manner. Due to the dependency of PRMT5 on cofactors for biological function, targeting a protein-protein interaction presents a promising therapeutic model for development of specific and selective therapeutic compounds. Three recent reports establish a PRMT5 substrate adapter binding motif and subsequent development of an inhibitor targeting said motif to disrupt PRMT5:RIOK1 interaction, though the clinical implication of these inhibitors remain unclear. Because PRMT5 represents a valuable therapeutic target with several Phase I clinical trials currently underway in solid and blood cancers, and because PRMT5 is the only PRMT of 9 family members that appears to require a cofactor (MEP50) and/or other factors for function, targeting the PRMT5:MEP50 protein-protein interaction may offer a specific approach as opposed to the catalytic or pan-MT inhibitors.

It has recently been found that PRMT5 can regulate target gene expression in both MEP50-independent and MEP50-dependent manners. Development of inhibitors targeting the PRMT5:MEP50 interaction may avoid potential non-specific targeting of other methyltransferases that utilize SAM as a cofactor. Such inhibitors may also enable selection of a specific type or stage of cancer that is dependent on the function of PRMT5:MEP50.

### SUMMARY

It has been discovered that protein arginine methyltransferase 5:methylosome protein 50 (PRMT5:MEP50) protein-protein interaction can be inhibited by compounds disclosed herein. It has also been discovered that the compounds are useful for the treatment of disease sensitive to PRMT5:MEP50 protein-protein interaction such as cancer.

In one aspect, provided is a compound of the formula

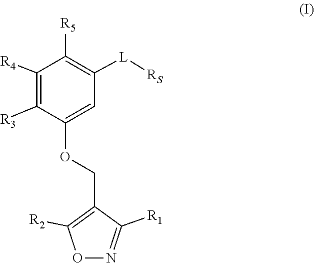

or a salt, hydrate, or solvate thereof;
wherein
   R₁ and R₂ are independently selected from the list consisting of alkyl, aryl, arylalkyl, alkenyl, cycloalkyl, heteroalkyl, heteroaryl, heteroarylalkyl, heteroalkenyl, and heterocycloalkyl, each of which is optionally substituted;
   R₃, R₄ and R₅ are each independently hydrogen or —(CH₂)ₓZ^{X}, where x is an integer from 0-6 and Z^{X} is halogen, hydroxy, C₁-C₆ alkanoyloxy, optionally substituted aroyloxy, C₁-C₆ alkyl, C₁-C₆ alkoxy, C₃-C₈ cycloalkyl, C₃-C₈ cycloalkoxy, C₂-C₆ alkenyl, C₂-C₆ alkynyl, C₁-C₆ haloalkyl, C₁-C₆ haloalkoxy, C₃-C₈ halocycloalkyl, C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, nitro; —CO₂R₆, or —CONR₇R₈, where R₆, R₇, and R₈ are each independently selected in each instance from hydrogen, C₁-C₆ alkyl, aryl-C₁-C₆ alkyl, and heteroaryl-C₁-C₆ alkyl or R₇, R₈, and the nitrogen to which they are attached form an optionally substituted heterocycle;
   L is a linker;
   R_{S} is cycloalkyl, aryl or heteroaryl, each of which is optionally substituted; or R_{S} is —NR₉R₁₀, in which R₉ and R₁₀ are each independently selected from hydrogen, C₁-C₆ alkyl, aryl-C₁-C₆ alkyl, and heteroaryl-C₁-C₆ alkyl; or R₉, R₁₀, and the nitrogen to which they are attached form an optionally substituted heterocycle.

In one illustrative embodiment, the compound, or a salt, a hydrate, or a solvate thereof of the preceding or any following compound is not

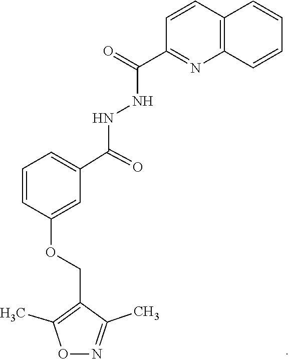 [SMILES: Cc1noc(C)c1COc1cccc(C(=O)NNC(=O)c2ccc3ccccc3n2)c1]

In one example compound of Formula (I), or a salt, a hydrate, or a solvate thereof, the linker (L) is C(O)NHNHC(O). In the same or a different example, R_{S} is quinolinyl. In either or both examples, or in a second different example, each of R₃, R₄ and R₅ is a hydrogen, and each of R₁ and R₂ is an alkyl.

In another example of the compound of Formula (I), or a salt, a hydrate, or a solvate thereof, the linker (L) is selected from the group consisting of C(O)(NH)_{N}C(O), where N is 1 or 2; C(O)NHNHSO₂, C(O)NH(CH₂)_{M}NHC(O), where M is an integer from 1 to about 6; C(O)NH(CH₂)_{M2}NHSO₂, where M2 is an integer from 1 to about 6; C(O)NH(CH₂)_{M3}, where M3 is an integer from 1 to about 6, and SO₂NH(CH₂)_{M4}, where M4 is an integer from 1 to about 6; HNC(O)(CH₂)_{M4}C(O)NH, where M4 is an integer from 0 to about 4; and

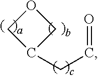

where a and b are independently 0, 1, or 2, with the proviso that a and b cannot both be 0, and c is an integer from 1 to about 4.

In a second aspect, pharmaceutical compositions containing one or more of the compounds of formula (I) or salts, hydrates, or solvates thereof are described herein.

In one example pharmaceutical composition, the linker (L) of the compound of Formula (I), or a salt, a hydrate, or a solvate thereof, is C(O)NHNHC(O). In the same or a different example, R_{S} of the compound of Formula (I), or a salt, a hydrate, or a solvate thereof, is quinolinyl. In either or both examples, or in a second different example, each of R₃, R₄ and R₅ of the compound of Formula (I), or a salt, a hydrate, or a solvate thereof, is a hydrogen, and each of R₁ and R₂ is an alkyl.

In another example of the compound of Formula (I), or a salt, a hydrate, or a solvate thereof, the linker (L) is selected from the group consisting of C(O)(NH)_{N}C(O), where N is 1 or 2; C(O)NHNHSO₂; C(O)NH(CH₂)_{M}NHC(O), where M is an integer from 1 to about 6; C(O)NH(CH₂)_{M2}NHSO₂, where M2 is an integer from 1 to about 6; C(O)NH(CH₂)_{M3}, where M3 is an integer from 1 to about 6; and SO₂NH(CH₂)_{M4}, where M4 is an integer from 1 to about 6; H4NC(O)(CH₂)_{M4}C(O)NH, where M4 is an integer from 0 to about 4; and

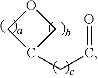

where a and b are independently 0, 1, or 2, with the proviso that a and b cannot both be 0, and c is an integer from 1 to about 4.

In one embodiment, the compositions include a therapeutically effective amount of the one or more compounds of formula (I) or salts, hydrates, or solvates thereof, for treating a patient with a cancer, including one or more of the foregoing example compounds. It is to be understood that the compositions may include other components and/or ingredients, including, but not limited to, other therapeutically active compounds, and/or one or more carriers, vehicles, diluents, adjuvants, excipients, and the like, and combinations thereof.

In another embodiment, unit doses of the compounds of formula (I) or salts, hydrates or solvates thereof, and pharmaceutical compositions containing one or more of the compounds or salts, hydrates or solvates thereof, are described herein. The unit doses include a therapeutically effective amount of the one or more compounds of formula (I) or salts, hydrates or solvates thereof, for treating a patient with cancer. The unit doses are in single or divided form, and may correspond to a daily dosage amount, or adjusted to a periodic amount that is shorter, including for multiple daily doses, or longer, including weekly or monthly doses. It is to be understood that the compositions may include other components and/or ingredients, including, but not limited to, other therapeutically active compounds, and/or one or more carriers, vehicles, diluents, adjuvants, excipients, and the like, and combinations thereof.

In another embodiment, methods of inhibiting protein arginine methyltransferase 5 (PRMT5) in a patient in need thereof include administering one or more of the compounds of formula (I) or salts, hydrates or solvates thereof, or compositions described herein. In another embodiment, methods for treating patients with cancer are also described herein, where the methods include administering one or more of the compounds of formula (T) or salts, hydrates, or solvates thereof, or compositions described herein to a patient with cancer. In another embodiment, the methods include administering a therapeutically effective amount of the one or more compounds of formula (I) or salts, hydrates or solvates thereof, and/or compositions described herein for treating patients with cancer. In another embodiment, uses of the compounds of formula (I) or salts, hydrates, or solvates thereof, and/or compositions described herein in the manufacture of a medicament for treating patients with cancer are described. In another embodiment, the medicaments include a therapeutically effective amount of the one or more compounds of formula (I) or salts, hydrates or solvates thereof, and/or compositions for treating a patient with cancer.

It is to be understood herein that the compounds, compositions, unit doses, and methods described herein may be used alone or in combination with other compounds useful for treating cancer including those compounds that may be therapeutically effective by the same or different modes of action. In addition, it is to be understood herein that the compounds or salts, hydrates, or solvates thereof, described herein may be used in combination with other compounds that are administered to treat other symptoms of cancer.

In another embodiment, compounds are described herein that are prepared by the foregoing processes.

### BRIEF DESCRIPTION OF THE FIGURES

FIG. 1A are graphs showing the overall survival curve based on high/low stratified protein arginine methyltransferase 5 (PRMT5) (top row) and methylosome protein 50 (MEP50)/WDR77 (bottom row) expression in hepatocellular carcinoma (HCC), head/neck squamous cell carcinoma (HNSCC), and pancreatic ductal adenocarcinoma (PDAC) from GEO/EGA/TCGA database.

FIG. 1B is a graph showing a scatter plot of the expression correlation of PRMT5 and MEP50/WDR77 in >1300 cell lines (Broad Institute).

FIG. 1C is a graph showing a scatter plot showing gene dependency (lethality of gene loss) correlation between PRMT5 and MEP50/WDR77 genes in >1300 cell lines (Broad Institute).

FIG. 2A is an illustration of the crystal structure 4GQB of PRMT5:MEP50 showing heterooctameric 4:4 organization (left) as well as single PRMT5:MEP50 heterodimer (right).

FIG. 2B is an illustration of MEP50 residues shown are in close interaction with the surface (R52, D99, D126) or buried inside (W54) the TIM barrel of PRMT5.

FIG. 2C is an illustration of MEP50 residues D99 and W54 occupy 7-angstrom wide pocket in TIM barrel of PRMT5 formed by a ridge consisting of PRMT5 H47 and R49.

FIG. 2D is a schematic representation of example BiFC constructs and mutations designed to study electrostatic interactions of PRMT5:MEP50 PPI (VN, venus N-terminus).

FIG. 2E is a schematic representation of example BiFC constructs and mutations designed to study electrostatic interactions of PRMT5:MEP50 PPI (VC, venus C-terminus).

FIG. 2F is a graph showing quantified BiFC efficiency of mutations in PRMT5, where **** is p<0.0001.

FIG. 2G is a graph showing quantified BiFC efficiency of mutations in MEP50, where ** is p<0.01, *** is p<0.001, **** is p<0.0001.

FIG. 2H is a graph showing FastContact Binding Energy Prediction of PRMT5 and MEP50 residues used in mutant screen. BiFC efficiency means are average of at least three biological replicates.

FIG. 3A are illustrations of all ≤4 angstrom contacts for residues PRMT5 R49 (left), MEP50 E276 (middle), MEP50 D99 (right) with waters in crystal structure 4GQB shown as spheres.

FIG. 3B is a table ranking predicted binding energy for top 11 residues mediating PRMT5:MEP50 protein:protein interaction for PRMT5.

FIG. 3C is a table ranking predicted binding energy for top 11 residues mediating PRMT5:MEP50 protein:protein interaction for MEP50.

FIG. 3D is a graph showing the ranked order for binding energy listed in FIG. 3B.

FIG. 3E is a graph showing the ranked order for binding energy listed in FIG. 3C.

FIG. 4A are images fluorescing Cerulean fluorescent protein (CFP) and Venus/Yellow fluorescent protein (YFP) images from BiFC mutant interaction experiment acquired at 10×magnification.

FIG. 4B are western blots showing Myc-tagged PRMT5(TIM) (Myc-VN-PRMT5) wild type (WT) or Mutants (Mts), HA-tagged MEP50 (HA-VC-MEP50) wild type (WT) or mutants (Mts), HA-tagged Cerulean (HA-Cerulean) fusion protein expression via western blot.

FIG. 5A is a depiction of the structural formulae of compounds 1-12 identified in a ZINCPharmer/SMINA virtual screen.

FIG. 5B is a graph showing ranked-order % inhibition of PRMT5:MEP50 for each of compounds 1-12 of FIG. 5A and a dimethylsulfoxide (DMSO) control based a BiFC-based interaction screen of COS-1 cells following 18-hour treatment at a 10 μM dosage.

FIG. 5C is a graph showing mean dose-response of BiFC efficiency of three biological replicates of compound 8 (Cpd 8) at doses of 0.25 μM, 0.5 μM, 1 μM and 5 μM as measured by % inhibition of PRMT5:MEP50 interaction, where ** is p<0.01*** is p<0.001.

FIG. 5D is a graph showing ranked-order inhibition in BiFC screen of compound 8 (Cpd 8) dose response as shown in FIG. 5C.

FIG. 5E is an illustration of the computational docking of compound 8 (Cpd 8) into the TIM barrel of PRMT5 where MEP50 W54 residue normally occupies. Left, PRMT5 and MEP50 shown with compound 8 occupying the MEP50 W54 binding pocket in PRMT5 TIM barrel; Middle left, expanded view; Middle right, rotated view with MEP50 removed, compound 8 docking position shows isoxazole ring solvent exposed and hydrogen bonding to PRMT5 R49, R68, and P44 backbone; Right, quinoline ring buried inside MEP50 W54 binding pocket of PRMT5 TIM barrel. BiFC Screens are single replicate. BiFC Efficiency means are average of at least three biological replicates.

FIG. 6A shows a scheme (1) for synthesis of compound 8b and example hydrazide analogs 13-16.

FIG. 6B shows a scheme (1) for synthesis of example hydrazide analogs 17-20.

FIG. 6C shows a scheme (2) for synthesis of example compound 8b analogs with imide linker (24) or amide linkers with various length (21-23, 25, 26).

FIG. 6D shows a scheme (3) for synthesis of example compound 8b analogs of Formula (Ia) with 3,3-disubstituted oxetane linkers.

FIG. 6E shows Scheme 4 synthesis of example compound 8b analogs of Formula 1(b) having oxalyl amide linkers.

FIG. 6F shows example analogs of Formula (Ib) of FIG. 6E.

FIG. 7A is a graph showing ranked-order results of BiFC screen of compounds 8a, 8b, 13, 14, 16, 17, 21, 22, 24, and 26 at 500 nM dose in COS-1 cells as a % Inhibition of the PRMT5:MEP50 protein-protein interaction.

FIG. 7B is a graph showing ranked-order results of BiFC screen of compounds 8a, 8b, 15, 17, 19, and 20 at 250 nM dose in COS-1 cells as a % Inhibition of the PRMT5:MEP50 protein-protein interaction.

FIG. 7C is a western blot of coimmunoprecipitation (Co-IP) of PRMT5 protein in LNCaP cell lysate following treatment with either DMSO or compound 17(Cpd 17) representative blot.

FIG. 7D is a graph showing integrated density of western blot Co-IP data from FIG. 7C across three biological replicates, where *** is p<0.001.

FIG. 7E is an illustration of computational docking of compound 8 derivative compound 17(Cpd 17), occupying same binding site along TIM barrel of PRMT5 as MEP50 W54 residue. The BiFC screens are single replicate, whereas co-immunoprecipitation quantitation means are average of three biological replicates.

FIG. 8A are images demonstrating that treatment of hormone naïve LNCaP cells with compound 17 (Cpd 17) at 250 nM, 500 nM, and 1000 nM for 72 hours resulted in both suppression of growth and induction of cell death in a dose dependent manner.

FIG. 8B is a graph showing respective mean IC₅₀ measurements of three biological replicants for each of compound 8b and compound 17(Cpd 17) in LNCaP cells over 72 hours treatment.

FIG. 8C are global histone H4R3 and H4R3me2s western blots from LNCaP cells treated with compound 17 (Cpd 17) at respective 250, 500, and 1000 nM doses over 72 hours.

FIG. 8D is a graph showing mean quantified densitometry from bands of the western blots of FIG. 8C for three biological replicants for compound 17 (Cpd 17), where *** is p<0.001.

FIG. 8E is a graph presenting expression analysis of PRMT5:MEP50-regulated Involucrine (IVL) gene in LNCaP cells treated with 500 nM compound 17 (Cpd 17) for 72 hours followed by RNA isolation and RT-qPCR, where * is p<0.05.

FIG. 8F is a graph presenting expression analysis of PRMT5:pICln-regulated androgen receptor (AR) in LNCaP cells treated with 500 nM compound 17 (Cpd 17) for 72 hours followed by RNA isolation and RT-qPCR, as measured by mean fold change of three biological replicants for compound 17 (Cpd 17), where ns is non-significant.

FIG. 8G is a graph presenting expression analysis of tumor suppressor genes tumor protein p53 (TP53), phosphatase and tensin homolog (PTEN), and RB transcriptional corepressor 1 (RB1) in LNCaP cells treated with 500 nM compound 17 for 72 hours followed by RNA isolation and RT-qPCR, where ns is non-significant, * is p<0.05.

FIG. 8H is a graph showing the IC₅₀ Curve for A549 (non-small cell lung cancer (NSCLC)) cell line treated with compound 17 (Cpd 17) for 72 hours.

FIG. 9A is a graph showing a Volcano plot of differentially expressed genes identified in LNCaP cells treated 72 hours with compound 17 (Cpd 17).

FIG. 9B is a Venn diagram of up-regulated genes (differentially expressed genes; DEGs) common between either PRMT5 or MEP50 KD in LNCaP cells and compound 17 (Cpd 17) treatment.

FIG. 9C is a Venn diagram of down-regulated genes (differentially expressed genes; DEGs) common between either PRMT5 or MEP50 KD in LNCaP cells and compound 17 (Cpd 17) treatment.

FIG. 9D is a graphic illustration of gene ontology enrichment of PRMT5-mediated differentiation/proliferation pathways following compound 17 (Cpd 17) treatment. GO terms shown are shared between compound 17 treatment and one or both of PRMT5/MEP50 knockdown in A549 cells and LNCaP cells; fold enrichment is shown as a heat map; and P values are shown as circle diameter.

FIG. 9E is a graphic illustration of gene ontology enrichment of PRMT5-mediated kinase/phosphatase activity and signaling/survival pathways following compound 17 (Cpd 17) treatment. GO terms shown are shared between compound 17 treatment and one or both of PRMT5/MEP50 knockdown in A549 cells and LNCaP cells; fold enrichment is shown as a heat map; and P values are shown as circle diameter.

FIG. 9F shows graphically presented Gene Set Enrichment Analyses of PRMT5-mediated TP53 signaling (plot: 1) and TGF-β signaling (plot: 2) based on total gene expression in compound 17 compared to DMSO samples.

FIG. 9G shows graphically presented Gene Set Enrichment Analyses of PRMT5-mediated kinase/phosphatase signaling (plot: 3) and development/differentiation (plot: 4) pathways based on total gene expression in compound 17 compared to DMSO samples.

FIG. 10 depicts a proposed model for compound 17 (Cpd 17) targeting of PRMT5:MEP50 and functional consequence, including suppressing differentiation/development, TGF-β signaling, dysregulation of kinase/phosphatase-mediated signaling, transcription, and resulting activation of T53 induced apoptosis.

FIG. 11 shows high-performance liquid chromatography (HPLC) purity of compound 17 yield.

### DETAILED DESCRIPTION

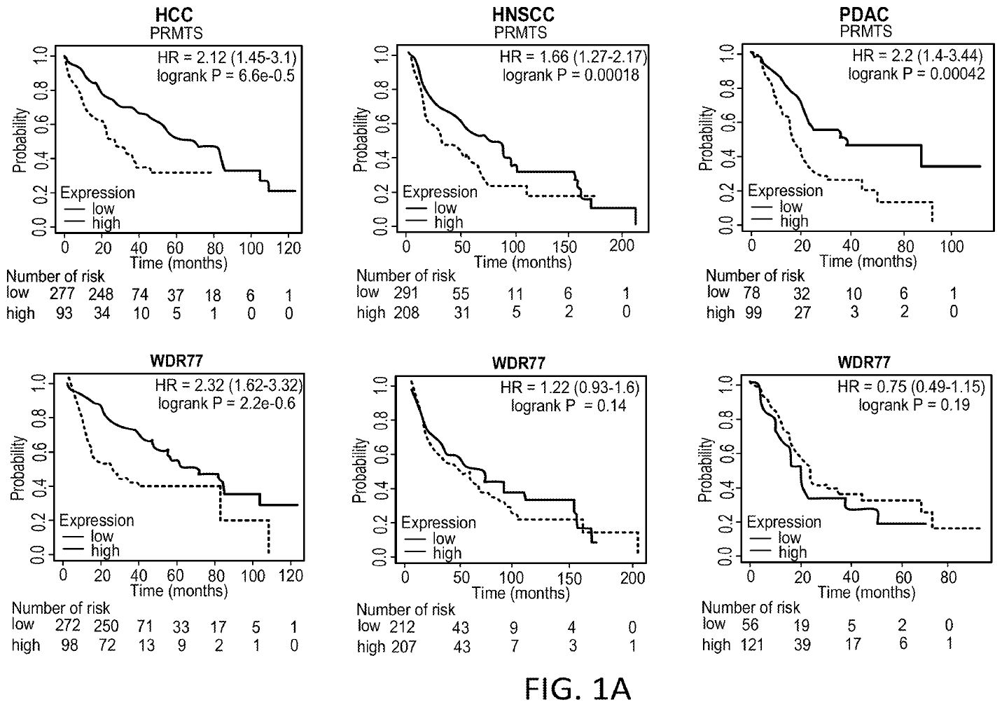

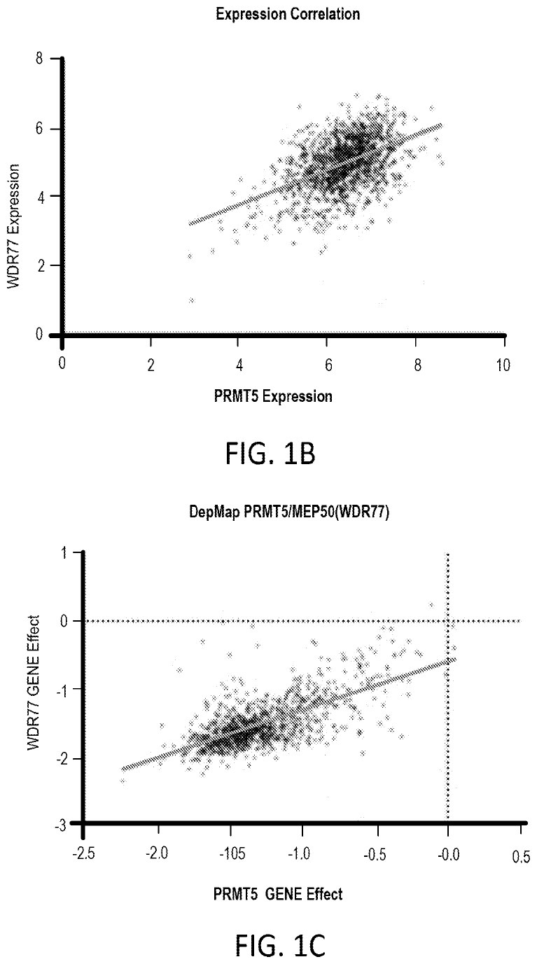

Clinical and in vitro data demonstrate that PRMT5 is frequently overexpressed in cancers and that its overexpression correlates with poor clinical outcome (FIG. 1A). Further, PRMT5 and MEP50 expression correlate positively in patient samples as well as cell lines derived from normal and cancerous tissue. In an analysis of over 1300 cell lines, PRMT5 correlated strongly with MEP50/WDR77 gene expression via the Cancer Dependency Map (FIGS. 1B, 1C). (Broad, DepMap 21Q3 Public, 2021, 14529771955 Bytes; see Dempster et al., preprint; Cancer Biology, 2019.)

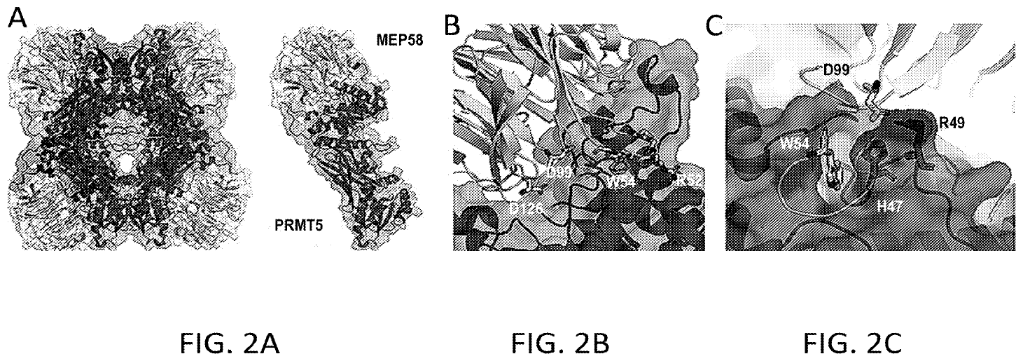

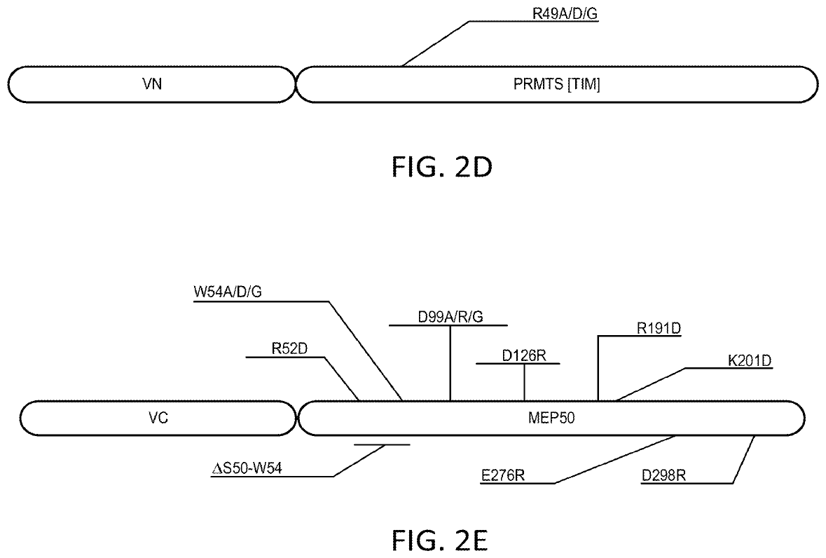

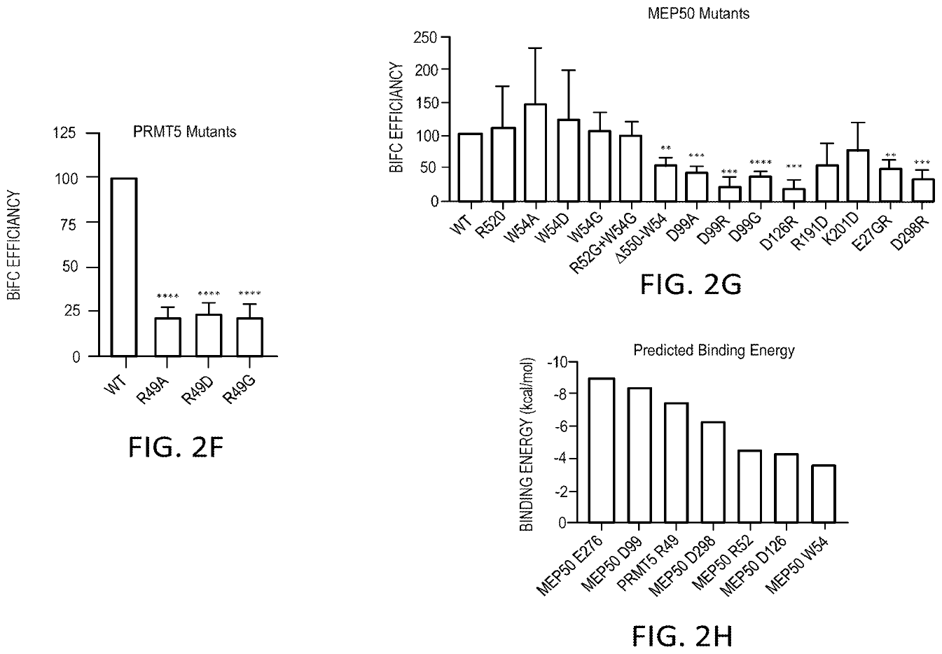

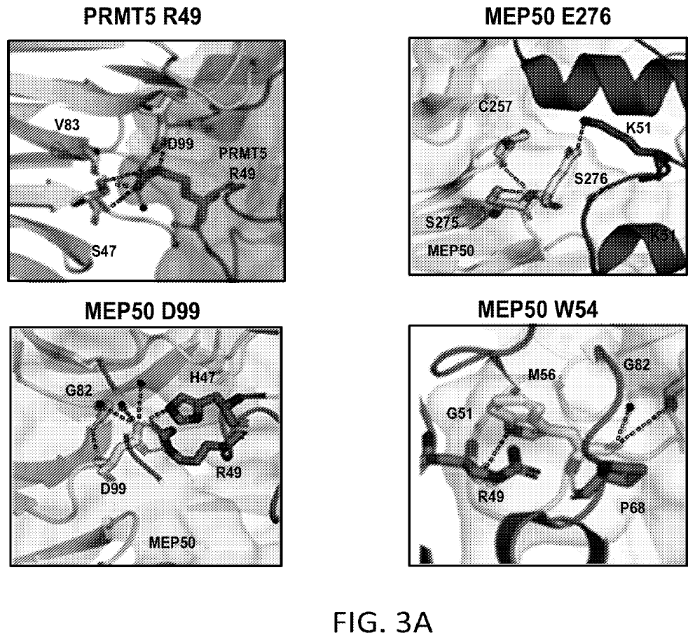

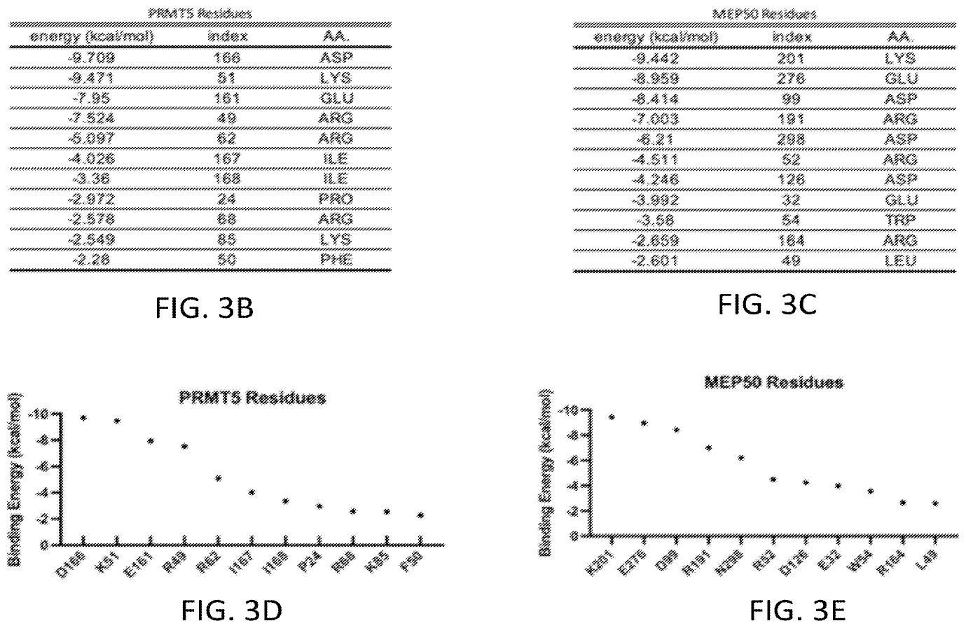

FIGS. 2A-C illustrate the PRMT5:MEP50 PPI Interface as a Druggable Target. PRMT5 forms a complex with MEP50 through its N-terminal TIM barrel domain (residues 1-292), and the interaction involves an interface completely occupying the bottom surface (with the top surface defined as the surface following directionality of the innermost beta strand) of the MEP50 protein (FIG. 2A). It was discovered by analyzing PRMT5:MEP50 crystallographic structure 4GQB from Protein DataBank (PDB) that inhibition of PRMT5 could be affected by inhibition of the protein-protein interaction of PRMT5 with MEP50. (Antonysamy et al., Proc. Natl. Acad. Sci. 2012, 109 (44), 17960-17965.) Referring to FIGS. 3A-3E, the protein-protein interaction interface was analyzed, and it was observed that electrostatic interactions present in the protein-protein interaction interface contribute to orientation and binding. Five residues that may play a role in mediating the interaction were identified. PRMT5 R49 extends from the TIM barrel and interacts with MEP50 D99 in the 2^{nd} β-propeller of MEP50 (FIGS. 2B, FIG. 2C). PRMT5 R49 also forms contacts with three co-crystallized water molecules as well as two contacts with MEP50 D99 and one contact each with MEP50 V83, S47 (FIG. 3A). MEP50 W54 is buried into a pocket of PRMT5, also in the TIM barrel, and appears to be involved in a stacking interaction with PRMT5 H47 (FIGS. 2C, 3A). Additionally, MEP50 R52 lies solvent exposed between two alpha helices in the PRMT5 TIM barrel, even though it does not participate in any hydrogen bonding. Collectively, these five residues represent potential electrostatic interactions that may be functionally evaluated to assess importance for the PRMT5:MEP50 protein-protein interaction. Using FastContact binding energy prediction software (v 2.0) developed by Champ et al. (Nucleic Acids Res. 2007, 35 (Web Server), W556-W560), 11 residues were identified on each of PRMT5 and MEP50 that were inferred to contribute to binding energy, as demonstrated in FIGS. 3B-3E.

Also identified were additional key residues to include in a bimolecular fluorescence complementation (BiFC)-based mutant screen. MEP50 D126 (contacts with PRMT5 N21 and co-crystallized water), MEP50 R191 (2 contacts with PRMT5 E161), MEP50 K201 (2 contacts with PRMT5 D166), MEP50 D298 (3 contacts with PRMT5 R62), and MEP50 E276 (one contact with PRMT5 K51 and one with co-crystallized water) were added based on this prediction. A BiFC assay was used to assess the feasibility of targeting the interaction via this interface. (See Hu et al., Mol. Cell 2002, 9 (4), 789-798, and Kodama et al. BioTechniques 2012, 53 (5), 285-298, each of which is incorporated herein in its entirety for its teachings regarding same.) BiFC is based on the proximity of two interacting proteins and has been used for visualization of protein-protein interactions in live cells and animals as well as for screening of protein-protein interactions. (See Kodama et al., In Methods in Cell Biology; Elsevier, 2013; Vol. 113, pp 107-121.) The VN (the N-terminal Venus fluorescent protein residues 1-154) were fused to the N-terminal PRMT5 TIM domain and VC (the C-terminal Venus fluorescent protein residues 155-238) were fused to the N-terminal end of MEP50, as illustrated in FIGS. 2D and 2E, respectively.

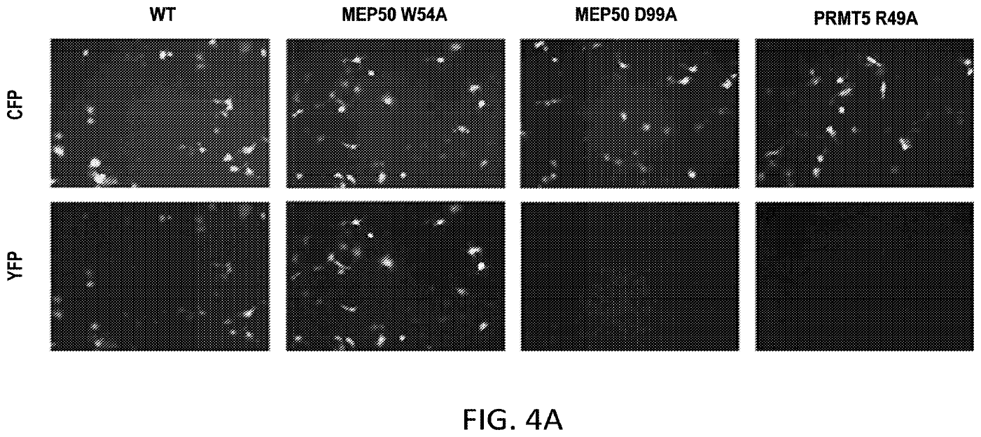

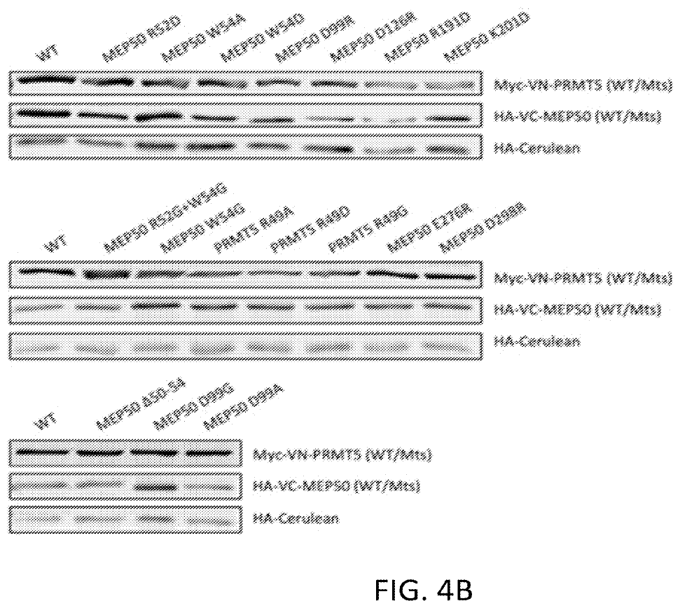

The mutations described above were introduced (PRMT5 residue R49A/D/G as well as MEP50 residues R52D, W54A/D/G, ΔS50-W54 deletion mutant, D99A/R/G, D126R, R191D, K201D, E276R, D298R), mutant expression was confirmed, and their interaction quantified via BiFC efficiency (see FIGS. 2F, 2G; FIGS. 4A, 4B). PRMT5 mutations R49A/G/D all resulted in suppressed BiFC efficiency, indicating decreased interaction between PRMT5 and MEP50 (FIG. 2F). Furthermore, seven MEP50 substitutions (ΔS50-W54, D99A/R/G, D126R, E276R, D298R) also resulted in a significant reduction of the BiFC efficiency (FIG. 2G). Mutations MEP50 D99A/R/G or PRMT5 R49A/G/D resulted in the strongest decrease of PRMT5:MEP50 interaction with over 70% decreased interaction. This appears to be consistent with disruption of a three-residue (MEP50 D99 and PRMT5 R49, in addition to PRMT5 H47) bridge mediated by at least four hydrogen bonds at the PRMT5:MEP50 protein-protein interaction interface, creating a 7-angstrom wide pocket between the bridge and PRMT5 R68 in which MEP50 W54 inserts (FIG. 2C). Further, MEP50 W54 (insertion residue) mutants W54A and W54D resulted in increased variability in BiFC assay, indicating that substitution may make MEP50 marginally more stable, but that MEP50 W54 burial into the PRMT5 TIM barrel pocket appear to be important to mediate PRMT5:MEP50 interaction. Individually, W54 and R52 mutants did not decrease binding, but deletion of first β-propeller loop residues S50-W54 reduced interaction by almost 50% (FIG. 2G). Western blot analysis confirmed that these mutations did not alter the expression and stability of BiFC fusion proteins (FIG. 4B). FastContact binding energy prediction also supported live cell BiFC data with MEP50 D99 and PRMT5 R49 contributing significant binding energy to the protein-protein interaction (FIG. 2H). These results suggested that targeting the electrostatic interactions at the interface of PRMT5:MEP50 protein-protein interaction may be achievable. Collectively, in silico prediction of binding energy and in vitro live-cell BiFC analysis with PRMT5 and MEP50 mutants were in agreement. Development of small molecules targeting protein-protein interaction, particularly the binding interface via MEP50 W50-W54/D99 and PRMT5 R49 was initiated.

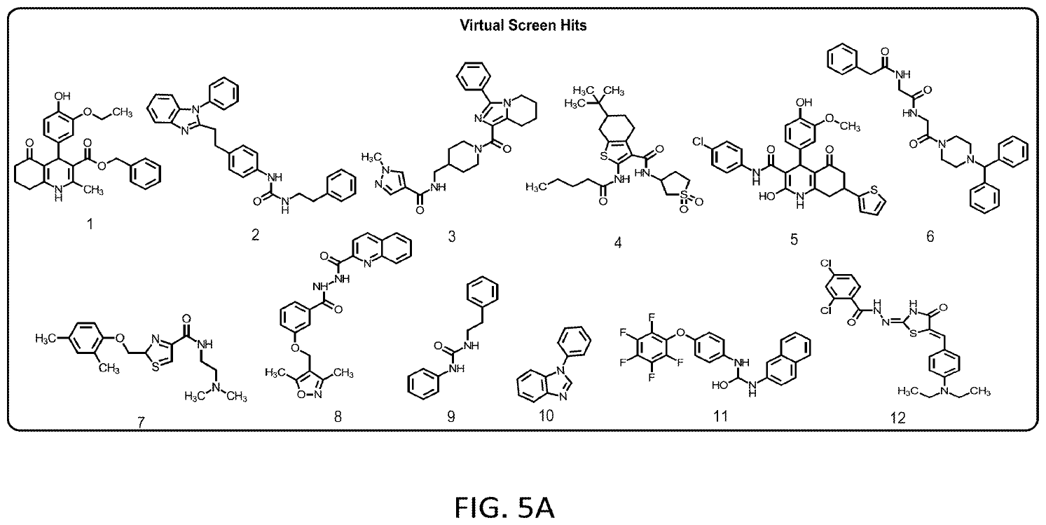

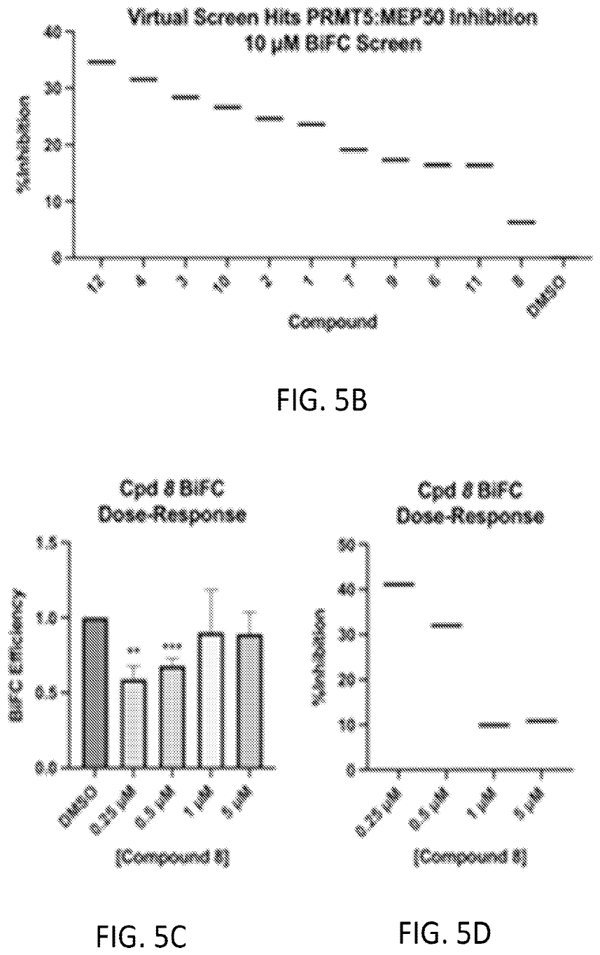

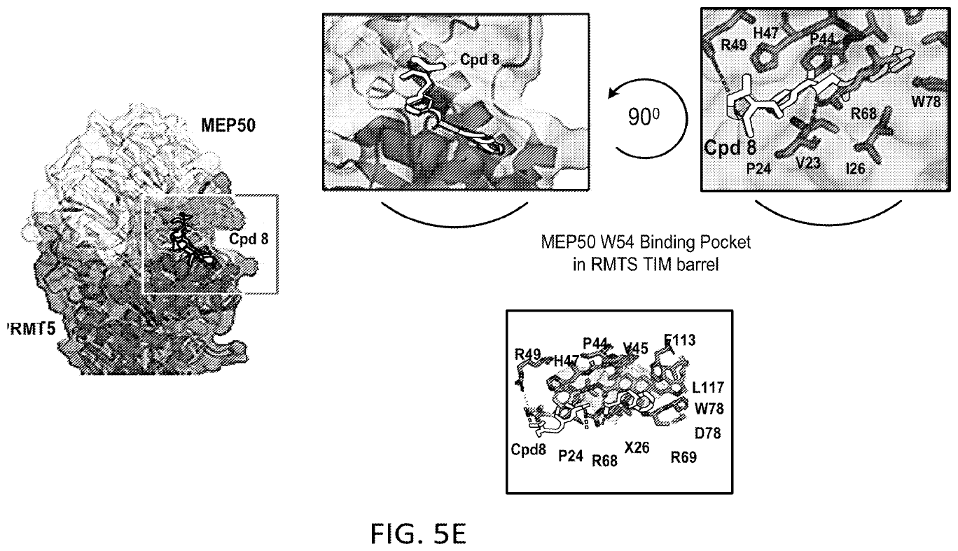

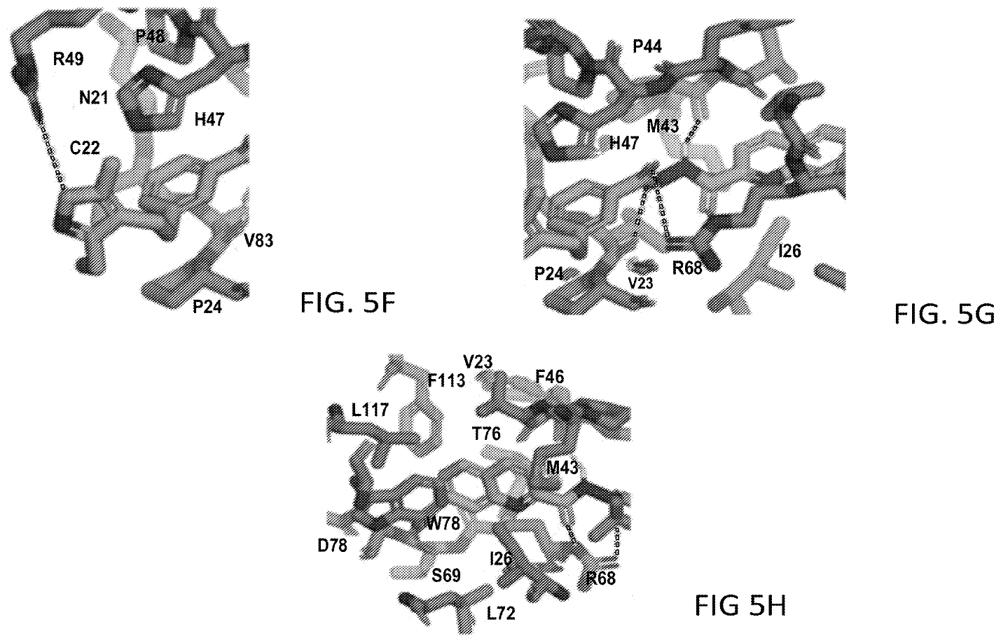

A virtual screen of close to 30 million small molecules from the ZINC database with the goal of disrupting the PRMT5:MEP50 interaction was performed using ZINCPharmer as described by Koes et al., Nucleic Acids Res. 2012, 40 (W1), W409-W414. Pharmacophore models addressing the hydrophobic pocket of MEP50 W54 that shows a stacking interaction with PRMT5 H47, as well as addressing hydrogen bonding patterns from crystal waters 811, 840, 935, 985 and 1002 in PDB 4GQB, were constructed. Compounds that fit the pocket were further minimized using SMINA, and those that remained in the pocket were selected for testing in vitro. Referring to FIG. 5A, twelve commercially available molecules selected based on the SMINA screen were purchased and a BiFC-based screen at 10 μM concentration was conducted. Compounds 12, 4, 3, and 10 showed the greatest inhibition at 24-35% (FIG. 5B). However, treatment of cells with compound 8 at 10 μM resulted in significant cell death, which is suboptimal for the BiFC assay and can prevent successful quantitation, suggesting the use of lower drug concentrations. To address this, a dose-response BiFC assay was performed, and it was found that compound 8 inhibited the BiFC efficiency in a dose-dependent manner (FIG. 5C). At 0.25 μM, compound 8 inhibited 41% of the BiFC efficiency (FIG. 5D). Compound 8 was identified as a promising hit via initial screen. Molecular docking indicated that compound 8 inhibits interaction of the W54 residue of MEP50 in a small pocket formed by TIM barrel loops 1 (C22-P24) and 2 (P44-H47 and T67-S69), in which the quinoline ring of compound 8 occupies the cavity mediating interaction with MEP50 W54 while the methyl-substituted isoxazole ring is exposed to solvent (in the absence of MEP50), occluding binding of MEP50 W54 into the TIM pocket (FIG. 5E). Additionally, docking suggests: 1) stacking interaction with PRMT5 H47, 2) hydrogen bonding with P44, R68, and 126 mediated by the hydrazide bond, and 3) hydrogen bonding with R49 mediated by the oxygen atom of the isoxazole ring (FIG. 5E, right). Collectively, these data demonstrate identification of compound 8 as a lead for further development of inhibitors of PRMT5:MEP50 protein-protein interaction due to interaction of the quinoline group with hydrophobic pocket of PRMT5 TIM barrel and solvent-exposed isoxazole ring participating in interaction with PRMT5 R49, together inhibiting MEP50 W54, D99 and PRMT5 R49 from contributing to protein-protein interaction.

### Synthesis of Compound 8 and its Analogs

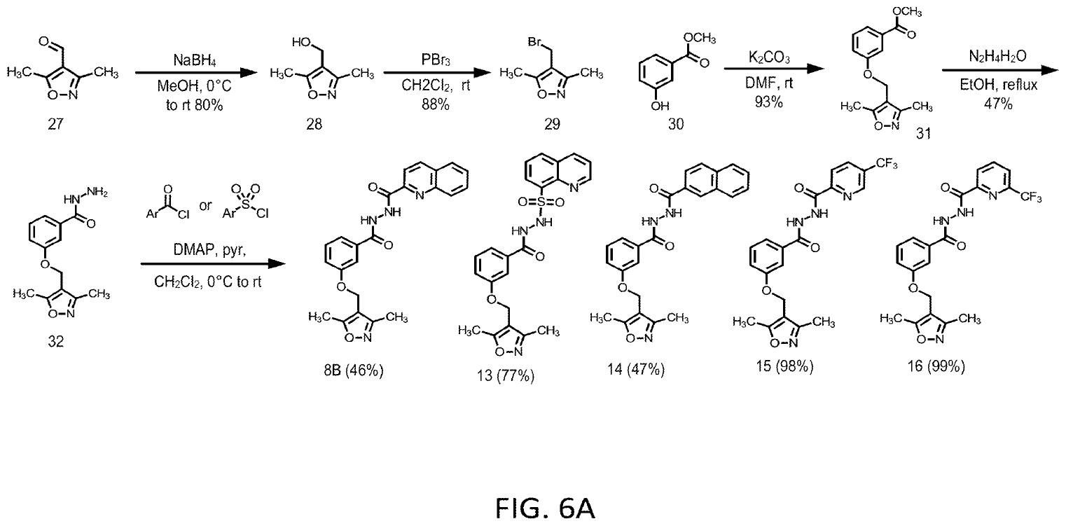

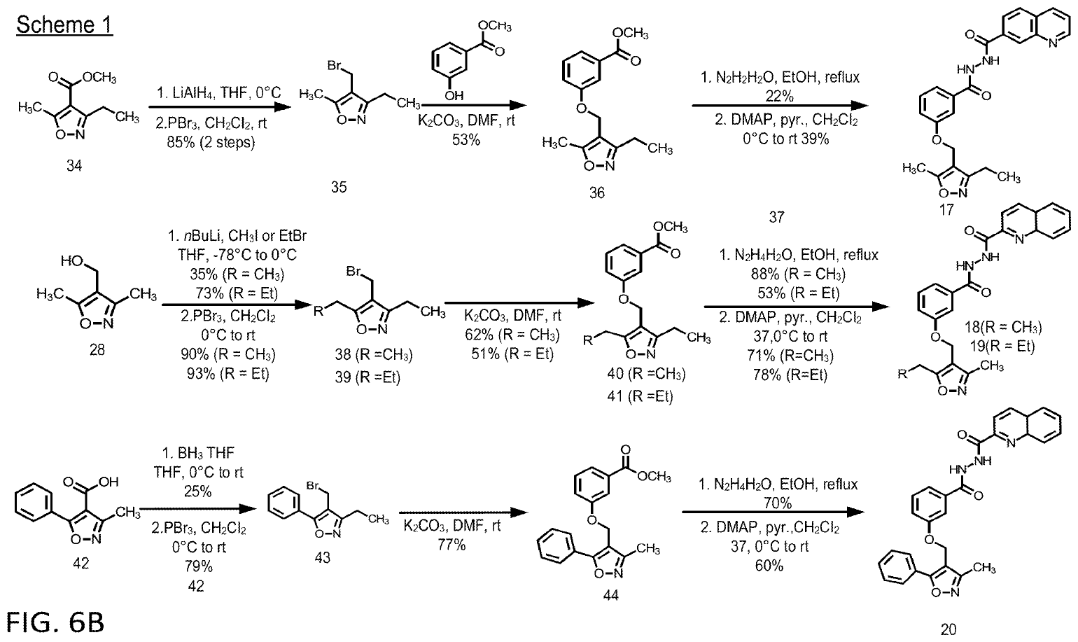

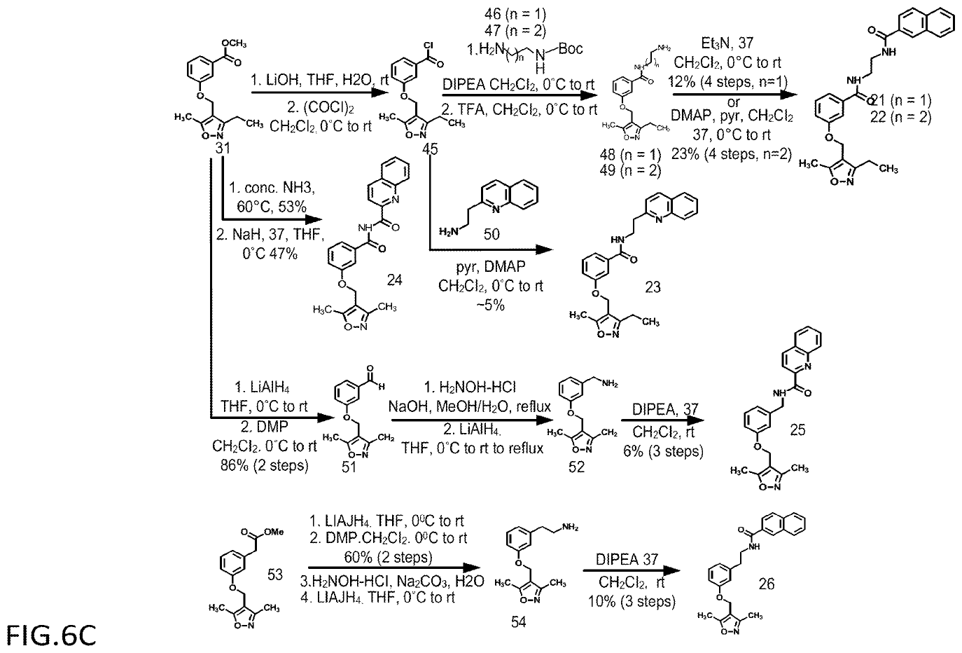

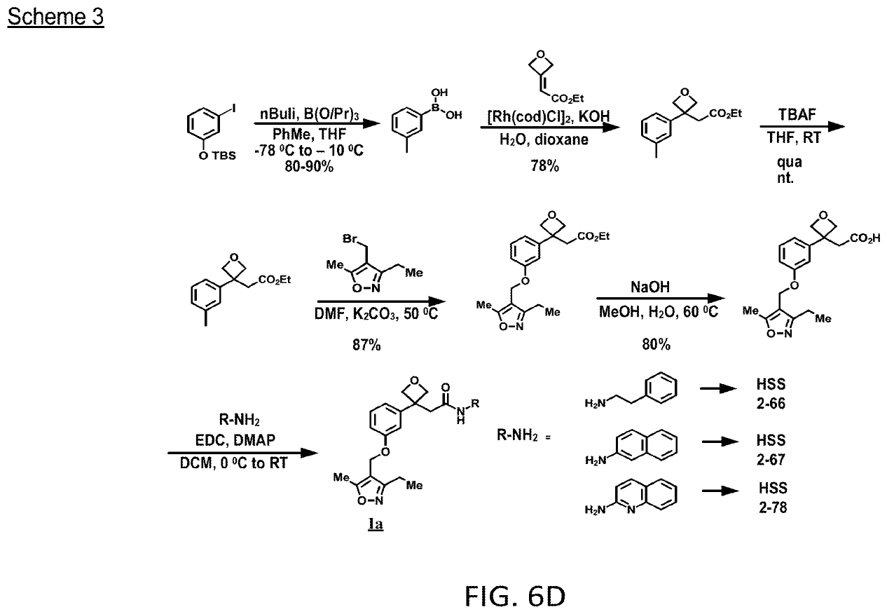

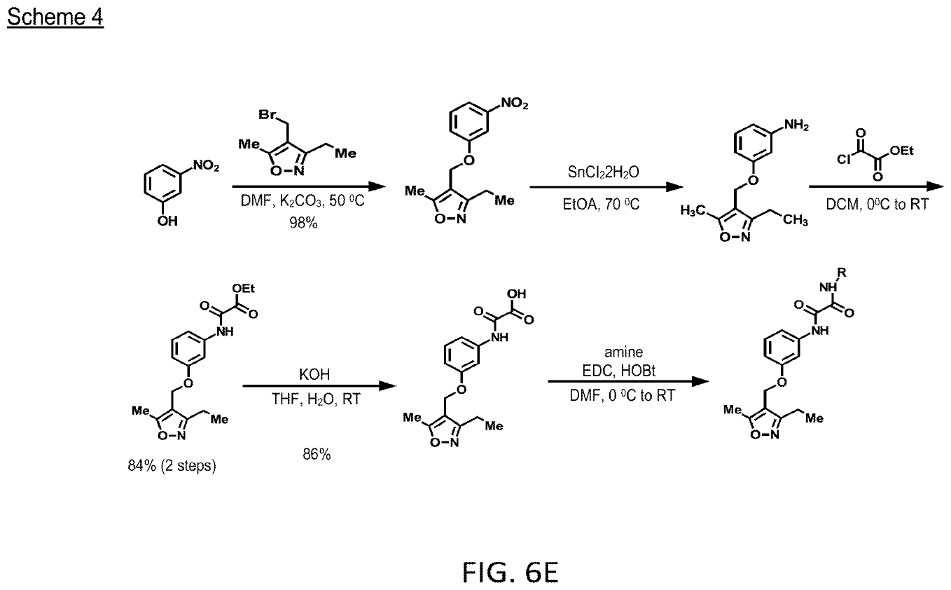

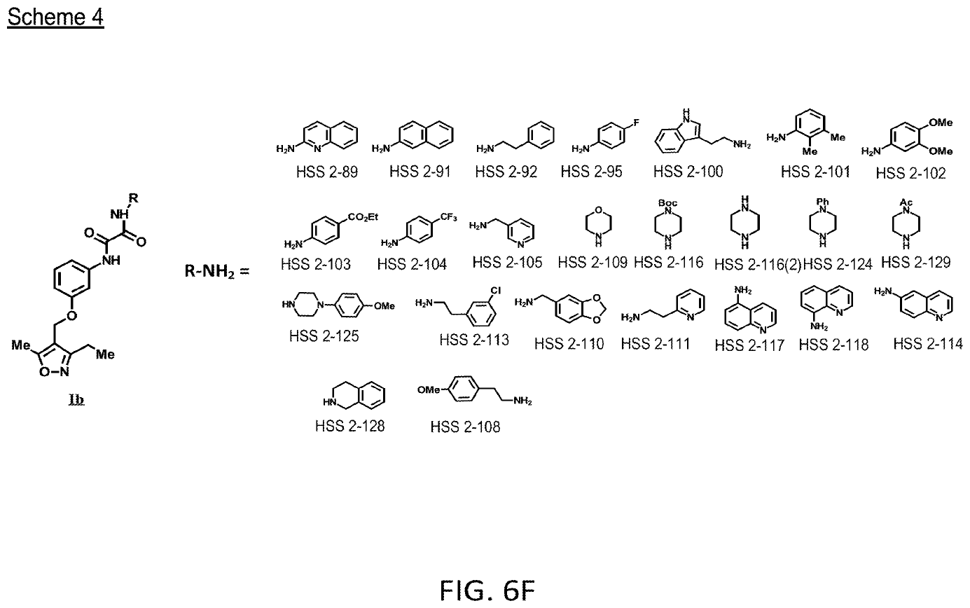

Based on the docking data, it was hypothesized that adding bulk to the quinoline group or extending the length of the methyl groups in the oxazole ring may facilitate greater affinity or occlusion of the binding site. Compound 8 was resynthesized (resynthesized compound is named as 8b; original compound 8 purchased from MolPort is listed as compound 8a) according to Scheme 1 of FIG. 6A. Its synthesis started from commercial starting material 27. Reduction of the aldehyde of 27 followed by bromination gave bromide 29 in high yield. Alkylation of phenol 30 with 29 afforded 31, which further reacted with hydrazine to provide hydrazide 32. Acylation of 32 with five different acyl chlorides (33) gave compound 8b and four of its analogs (13-16) with structural variation at the original quinoline group. Analogs with extended alkyl chain (17-19) or a phenyl group (20) at the C3 or C5 position of the isoxazole ring to facilitate hydrophobic interactions in the PRMT5 TIM barrel were synthesized as well by following a similar synthetic sequence.

Further rounds of example analogues for use as inhibitors of PRMT5:MEP50 protein-protein interaction were synthesized with (a) imide (24) or amide linkers with various length (21-23, 25, 26) according to Scheme 2 of FIG. 6C; (b) 3,3-disubstituted oxetane linkers according to Scheme 3 of FIG. 6D, and (c) oxalyl amide linkers according to Scheme 4 of FIGS. 6E and 6F.

### Identification of Compound 17 as Potent Analog of Compound 8

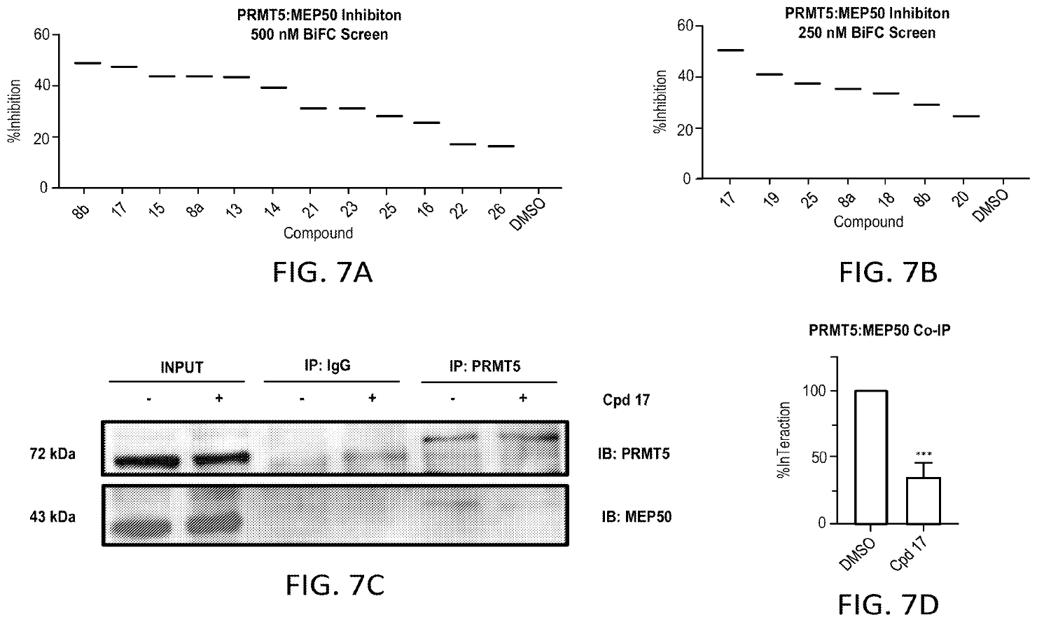

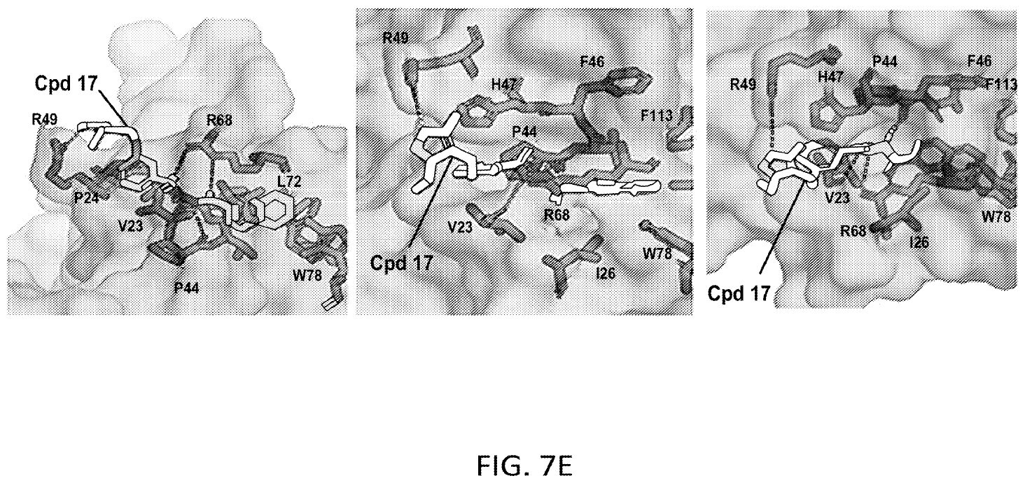

A BiFC screen as described above was performed, and it was observed that the top four inhibitors at 500 nM concentration were 8b, 17, 15, and 8a (FIG. 7A). These results suggested that the bulky groups (trifluorotolyl or quinyl) are desirable for binding and that an ethyl substitution at the C3 position on the isoxazole ring increases binding. Based on these observations, a second round of inhibitors was synthesized, in accordance with Scheme 2 of FIG. 6C, altering the hydrazide linker to imide (24) or amide with various length (21-23, 25, 26) to avoid potential pharmacokinetic-pharmacodynamic (PK/PD) issues associated with the hydrazide linker. A BiFC screen was performed at a lower concentration (250 nM), and it was determined that compound 17 was still the most effective at inhibiting PRMT5:MEP50 interaction (FIG. 7B). To assess functional protein-protein interaction inhibition in live cells following treatment with compound 17, co-immunoprecipitation (Co-IP) western blot was performed, and a 65.4% decrease in amount of MEP50 co-immunoprecipitated with PRMT5 bait across three independent biological replicates was observed (FIGS. 7C, 7D). When molecular docking simulation was performed on compound 17, as with compound 8, both compounds bound to the same pocket of PRMT5, but with the ethyl group of compound 17 extending further into the PRMT5:MEP50 protein-protein interaction (FIG. 7E). Interestingly, of all compounds synthesized, the most potent compound was highly similar to the initial hit, highlighting the importance of A) the hydrogen bond between PRMT5 R49 and the oxygen of the isoxazole ring, B) the hydrophobic interaction of the quinoline group with the PRMT5 TIM barrel pocket, and C) electrostatic/hydrogen bonding interactions of the hydrazide linker. Because live cell imaging (BiFC) demonstrated improved potency of compound 17, and Co-IP from cell lysates also suggested successful target engagement, biological characterization and functional confirmation in vitro utilizing prostate cancer cells were undertaken due to their dependence on PRMT5:MEP50 and prior validation of PRMT5 as a therapeutic target.

### Compound 17 Selectively Inhibits Prmt5:Mep50 Biological Function in Prostate Cancer Cells

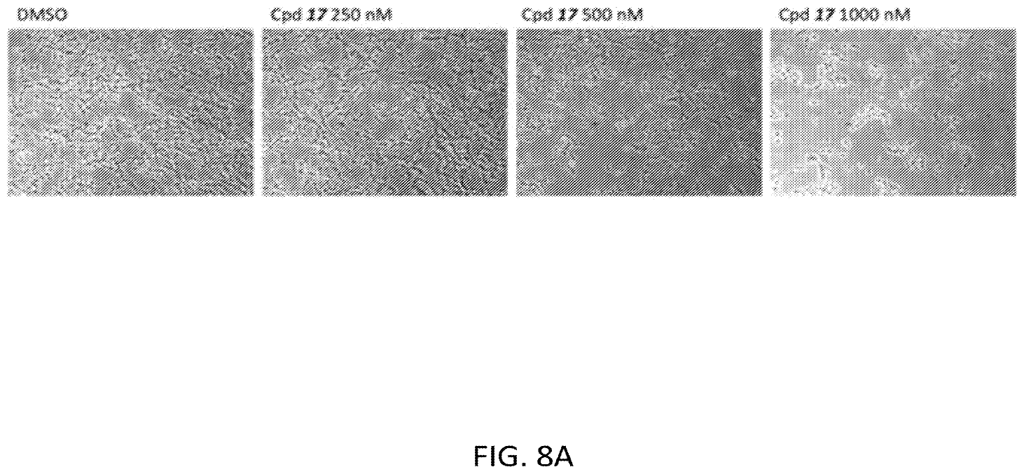

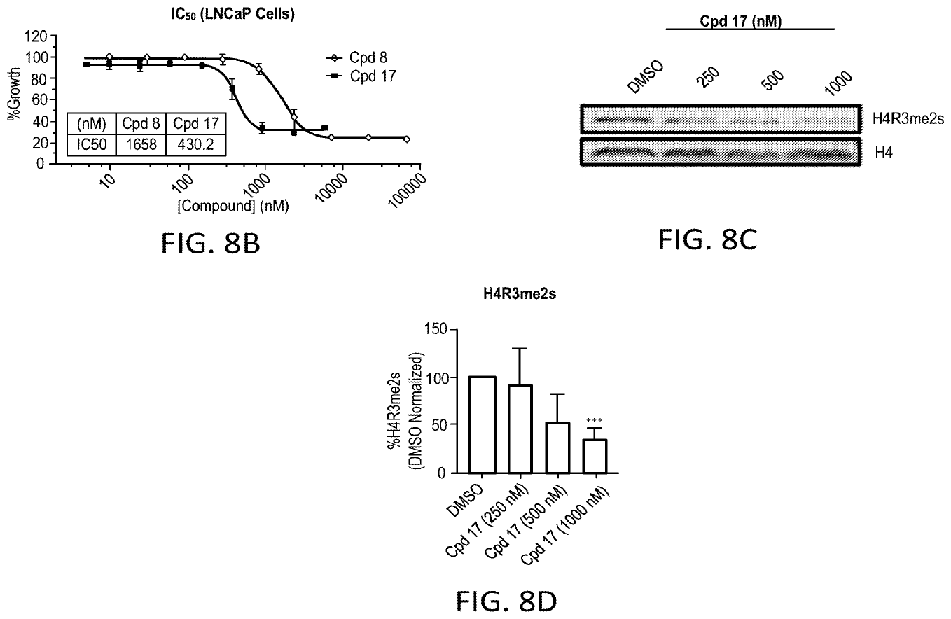

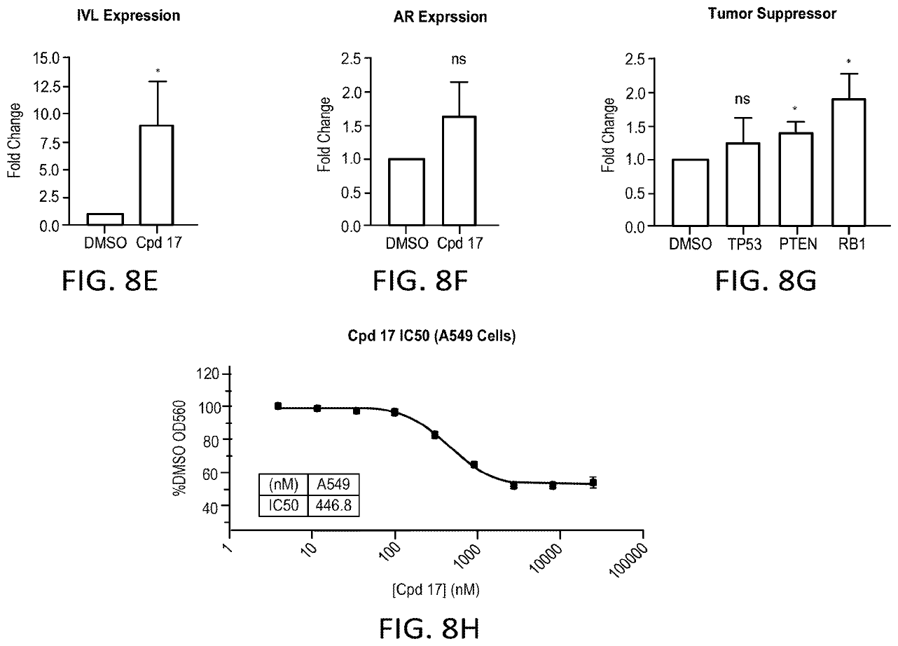

To evaluate the biological effects of compound 17 in cells, prostate cancer cells as an in vitro model system were used, as the roles of both PRMT5 and MEP50 have been studied previously. (See Owens et al., Mol. Cancer Ther. 2022, molcanther.MCT-21-0103-A.2021.). Treatment of hormone naïve LNCaP cells with compound 17 at 250 nM, 500 nM, and 1000 nM for 72 hours resulted in both suppression of growth and induction of cell death in a dose-dependent manner (FIG. 8A). The IC₅₀ of compound 17 was also calculated in LNCaP cells to be 430 nM when treated over 72 hours, compared with 1658 nM for lead compound 8b, a roughly 4-fold improved potency (FIG. 8B). As PRMT5:MEP50 are responsible for symmetric dimethylation of arginine 3 residue of Histone H4 (H4R3me2s), global level of H4R3me2s in LNCaP cells after treatment with compound 17 over 72 hours (FIG. 8C) was examined. It was observed that global H4R3me2s decreased by 65% after treatment with compound 17, and that global H4R3me2s levels decreased in a dose-dependent manner (FIG. 8D). PRMT5:MEP50 occupies the promoter region of the Involucrine (IVL) gene and was shown to repress IVL transcription in LNCaP and other cells. (Owens et al., iScience 2020, 23 (1), 100750; Saha et al., J. Invest. Dermatol. 2016, 136 (1), 214-224.) By contrast, PRMT5 and pICln (notably not MEP50) were shown to activate AR transcription. (Beketova et al., Cancer Res. 2020, 80 (22), 4904-4917.) Thus, the combination of IVL de-repression with unaltered AR expression serves as an ideal model system to evaluate the target engagement and selectivity of compound 17. Toward this end, LNCaP cells were treated with compound 17 for 72 hours, and a quantitative reverse transcription polymerase chain reaction (RT-qPCR) was performed to quantify the expression of both IVL and AR. Consistent with target engagement suggested by Co-IP result from LNCaP cells (FIG. 7D), compound 17 significantly de-represses PRMT5:MEP50-regulated IVL gene (as a positive control) without significantly altering expression of PRMT5:pICln-regulated AR gene (as a negative control) in LNCaP cells over 72 hours (FIGS. 8E, 8F), supporting the selective effect of compound 17 in cells in inhibition of PRMT5:MEP50 target gene IVL but no inhibition of PRMT5:pICln target gene AR. As it has been reported that PRMT5 regulates multiple tumor suppressor genes, including TP53, PTEN, and RB1, it was evaluated if treatment with compound 17 de-represses the transcription of these tumor suppressors in prostate cancer cells. Indeed, it was observed that compound 17 treatment caused upregulation of both PTEN and RB1 and to some extent TP53, albeit statistically insignificant (FIG. 8G). (See Chung et al., J. Biol. Chem. 2013, 288 (49), 35534-35547; Banasavadi-Siddegowda et al., Oncogene 2017, 36 (2), 263-274.) Loss of histone methylation and de-repression of PRMT5:MEP50 target gene IVL support biologically on-target functional consequence of PRMT5:MEP50 protein-protein interaction inhibition. Further, the improved potency in BiFC screen (FIGS. 7A, 7B) was also observed in four-fold increase in potency of IC50 for LNCaP prostate cancer cells. Compound 17 was similarly effective against non-small cell lung cancer (NSCLC) cell line A549 with IC₅₀ 447 nM (FIG. 8H). Functionally, no change was detected in AR expression, which, as reported by Deng et al., is regulated not by PRMT5:MEP50, but by PRMT5:pICln. (Oncogene 2017, 36 (9), 1223-1231.) Collectively, these biologically functional data suggest that compound 17 is a potent and selective inhibitor of the PRMT5:MEP50 interaction.

### Compound 17 Treatment Targets Prmt5:Mep50-Mediated Cellular Functions

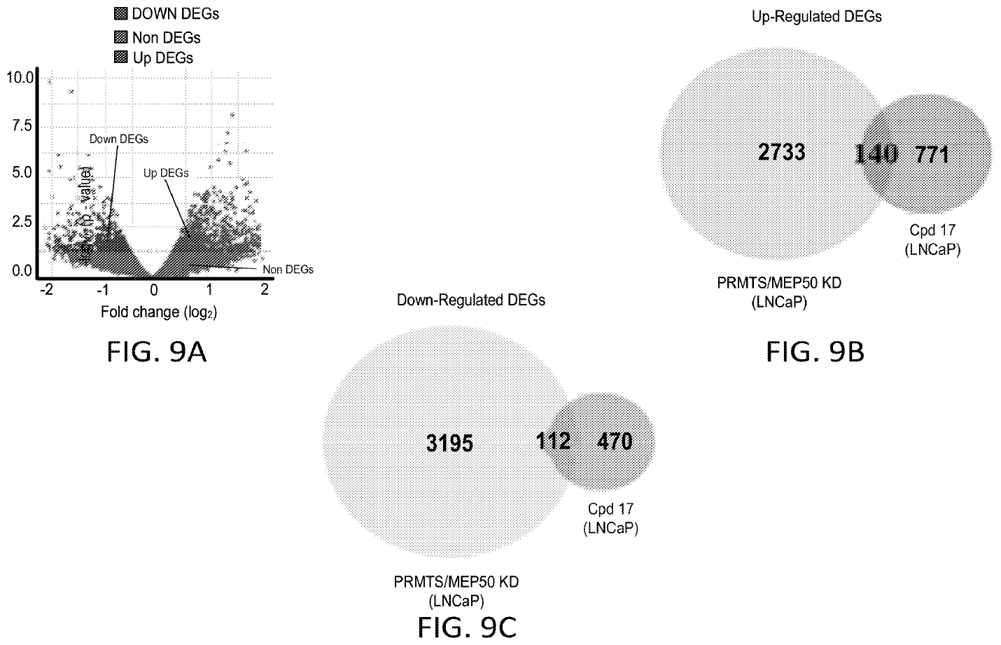

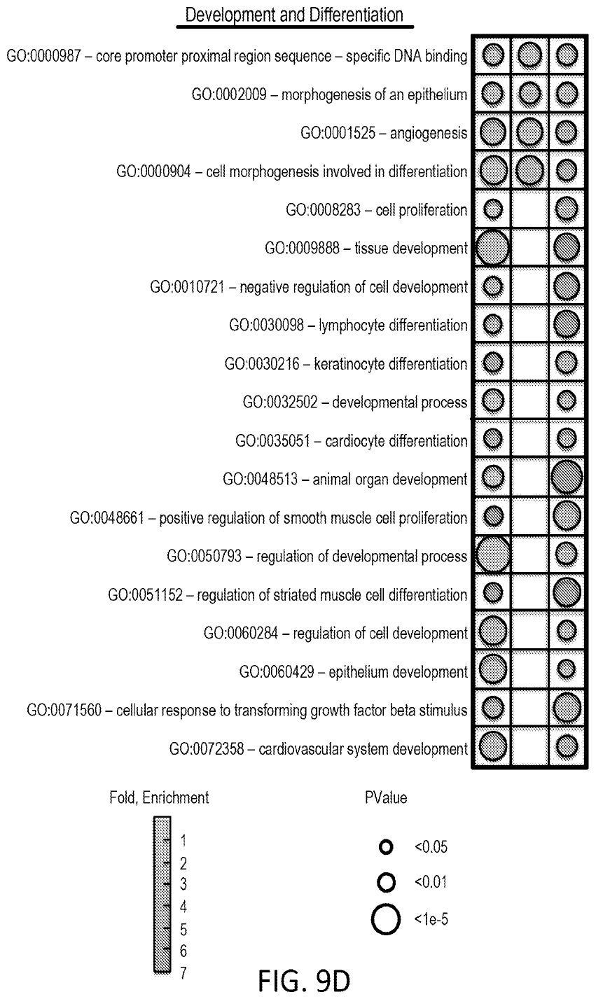

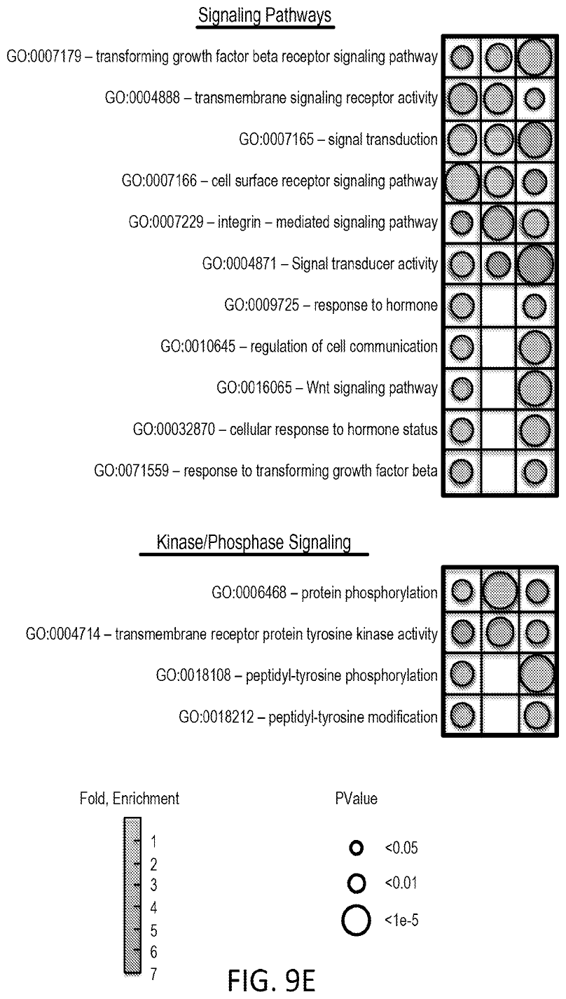

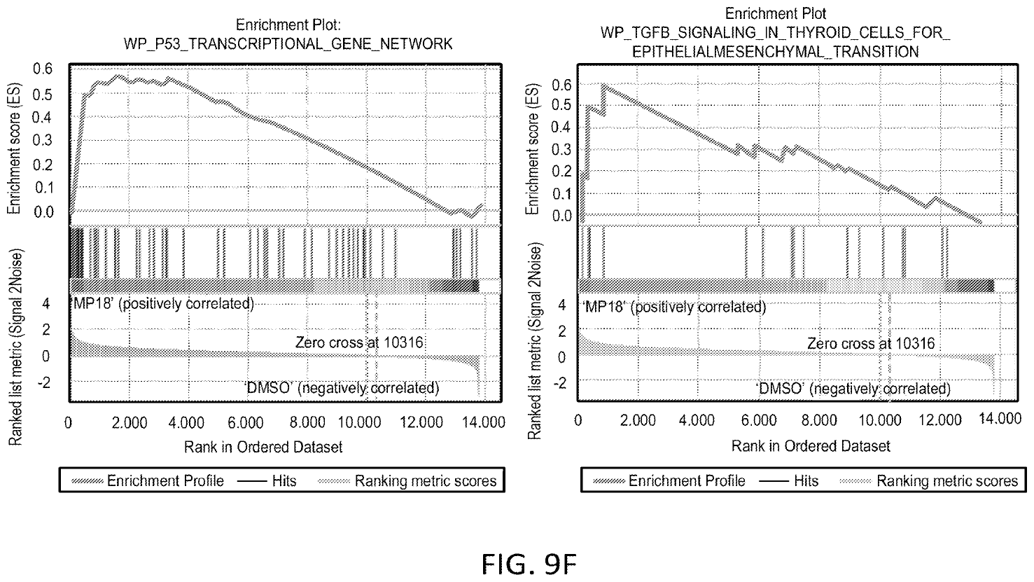

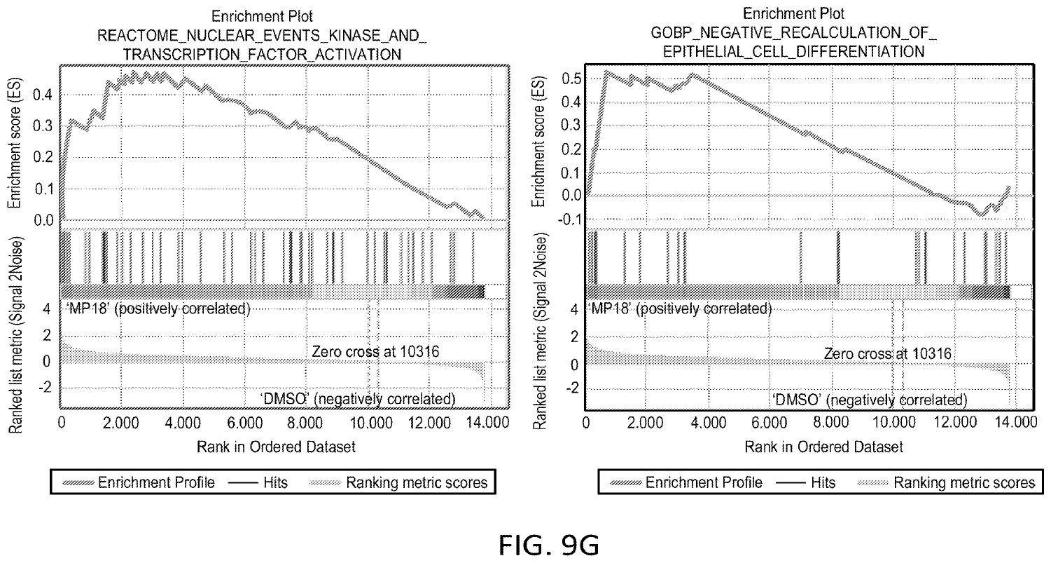

Because compound 17 targets PRMT5:MEP50 protein-protein interaction, it was hypothesized that similar dysregulation of genes between PRMT5 knockdown, MEP50 knockdown, and compound 17 treatment would be identified. To experimentally test this, RNA-seq in LNCaP cells treated with compound 17 over 72 hours was performed. Overall, 1,493 differentially expressed genes (DEGs) were identified between compound 17 treatment and DMSO control (FIG. 9A). Consistent with RNA-seq performed on samples with knockdown (KD) of PRMT5 or MEP30 in LNCaP cells, compound 17 treatment did display a small degree of overlap with a core set of up- and down-regulated genes as was observed in PRMT5 or MEP50 KD alone (140 and 112, respectively) (FIGS. 9B, 9C). However, the degree of overlap between DEGs identified in compound 17 treatment versus PRMT5 or MEP50 KD was unexpectedly a minority of the 1493 total genes identified in the compound 17 treatment (911 up- and 582 down-regulated genes).

To evaluate the biological consequence of these overlapping genes, S comparative analysis was performed between compound 17 treated LNCaP cells, PRMT5/MEP30 KD in LNCaP cells, as well as PRMT5/MEP30 KD in A549 lung cancer cells by analyzing a publicly available dataset in which A549 NSCLC cell lines were also subjected to PRMT5 and MEP50 knockdown followed by RNA-seq. (Chen et al., Oncogene 2017, 36 (3), 373-386.) After performing differential expression analysis on each of the three datasets, GO enrichment was performed for the up- or down-regulated genes within each treatment (PRMT5 KD, MEP 50 KD, or compound 17 treatment). As PRMT5:MEP50 mediate multiple pathways in the cell, it was not surprising that certain GO terms were enriched in both up- and down-regulated genes. For this reason, all enriched GO terms agnostic of differential expression directionality were combined and enriched terms common to all data sets were identified (compound 17 treatment in LNCaP cells, PRMT5/MEP50 KD in LNCaP cells, and PRMT5 MEP50 KD in A549 cells (FIGS. 9D, 9E).

Broadly, compound 17 treatment and PRMT5/MEP50 knockdown showed commonly enriched pathways in three major pathways significant to the hallmarks of cancer, including differentiation/proliferation (FIG. 9D), kinase/phosphatase activity (FIG. 9E, bottom panel), and multiple signaling/survival pathways (FIG. 9E top panel). (See Hanahan, D. Cancer Discov. 2022, 12 (1), 31-46.) Significantly, enrichment of TGF-β signaling in all three datasets and wnt signaling shared between compound 17 and PRMT5/MEP50 knockdown in A549 cells was detected. Collectively, 27 enriched GO terms shared between all three datasets and 142 terms shared between compound 17 and at least one other dataset were identified, with all GO terms of Fold Enrichment >2 (FIGS. 9D, 9E). As complementary approach to GO enrichment, gene set enrichment analysis (GSEA) was used to include all genes identified in the compound 17 RNAseq data set. With reference to FIGS. 9F and 9G, overlap of similar pathways as observed in the GO enrichment was found, particularly with PRMT5-mediated TP53 signaling (plot: 1), TGF-β signaling (plot: 2), kinase/phosphatase signaling (plot: 3), as well as epithelial cell development/differentiation (plot: 4).

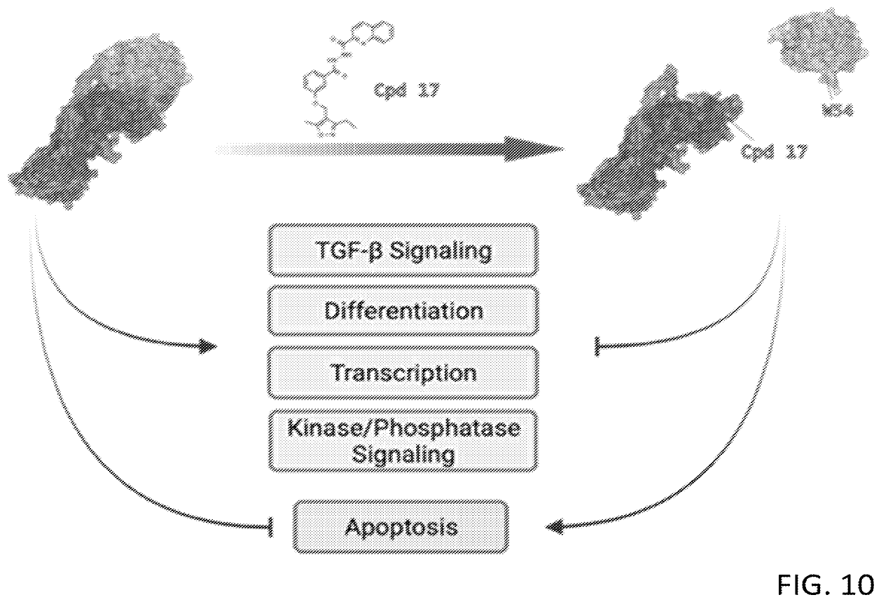

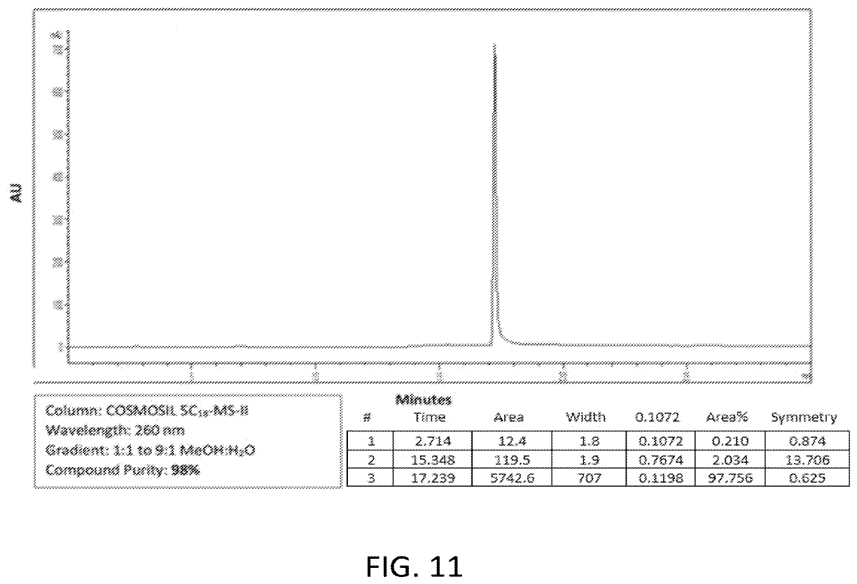

A comprehensive approach involving 1) differential expression and GO enrichment of compound 17 treated LNCaP cells, 2) comparative analysis of differential expression across PRMT5/MEP50 KD in LNCaP and A549 cells, and 3) whole-transcriptome analysis utilizing GSEA of compound 17 treated LNCaP cells was used. As FIGS. 9A-9G demonstrate, Compound 17 treatment resulted in dysregulation of multiple processes in which PRMT5 has been extensively characterized including chromatin structure and epigenetic regulation, proliferation/differentiation, MAPK/ERK signaling, and apoptosis/TP53 regulation, and shows significant overlap between PRMT5 and MEP50 knockdown in two independent cell lines. (See Deng et al., Oncogene 2017, 36 (9), 1223-1231; Jiang et al., Cancer Med. 2018, 7 (3), 869-882; Yin et al., Nat. Commun. 2021, 12 (1), 3444.) Prostate cancer cell line LNCaP and NCSLC cancer cell line A549 showed similar IC₅₀<450 nM. The data appear to implicate TGF-β signaling present in each of the PRMT5/MEP50 LNCaP knockdown, PRMT5/MEP50 A549 knockdown, and compound 17 LNCaP treatment datasets. Together, these data suggest that A) treatment with compound 17, a PRMT5:MEP50 protein-protein interaction inhibitor, results in similar biological functional consequence as knockdown of PRMT5 or MEP50 in multiple cell lines, B) compound 17 treatment is able to inhibit multiple PRMT5-regulated pathways critical to the survival and proliferation of lung and prostate cancer cells, and C) PRMT5:MEP50 protein-protein interaction inhibition via compound 17 may potentially inhibit the TGF-β signaling axis, which has been extensively characterized as a key driver in multiple solid tumor cancers and leukemias/lymphomas. Collectively, data support further refinement of lead compound 17 as a potential therapeutic inhibitor with specificity to PRMT5:MEP50-regulated targets and biological efficacy in inhibition of multiple hallmark pathways in cancer cells (FIG. 10).

PRMT5 has been validated as a therapeutic target in multiple cancers with ten active clinical trials as reported in clinicaltrials.gov. (See Hwang et al., Exp. Mol. Med. 2021, 53 (5), 788-808.) All compounds undergoing active trials are either SAM- or Substrate-competitive inhibitors. Given the multiple roles of PRMT5 in virtually all developing normal cells, the clinical applicability of these PRMT5 inhibitors remains unknown until such clinical trials are complete and adverse effect data become available. A recent approach by Shen et al. was to develop a proteolysis targeting chimera (PROTAC) molecule targeting PRMT5 via the SAM binding site to the VHL E3 ligase. (J. Med. Chem. 2020, 63 (17), 9977-9989.) A conserved PRMT5 binding motif (PBM) has been identified that mediates interaction with PRMT5 cofactors COPR5, RioK1, and pICln and an inhibitor has been developed by McKinney et al. to target the interaction of PBM with RioK1. (J. Med. Chem. 2021, 64 (15), 11148-11168.) This inhibitor also appears to be effective in suppressing the growth of MTAP-deleted cancer cells. Given the unique cofactor-dependency of PRMT5 among the PRMT family of proteins, we proposed targeting the protein-protein interaction interface directly between PRMT5 and MEP50 by occluding the MEP50 W54 binding pocket in the PRMT5 TIM barrel.

Our virtual screen and BiFC screens led to the identification of compound 8 as an initial hit. Further synthesis and screening of additional analogs resulted in the identification of compound 17 with almost 4-fold improvement in potency based on IC₅₀ in LNCaP cells. Significantly, we provided several pieces of evidence supporting that compound 17 is specific and on-target. Firstly, compound 17 decreased global histone H4R3me2s, an epigenetic mark mediated by PRMT5:MEP50. Second, treatment with compound 17 resulted in decreased repression of IVL gene normally repressed by PRMT5:MEP50, especially in non-keratinocyte cell types as reported by Saha et al. (J. Invest. Dermatol. 2016, 136 (1), 214-224), without affecting the expression of AR, which is regulated by PRMT5:pICln instead as further reported by Beketova et al. (Cancer Res. 2020, 80 (22), 4904-4917). Lastly, we utilized Co-IP to demonstrate decreased binding of endogenous MEP50 to endogenous PRMT5 in LNCaP cell lysate. Thus, compound 17 represents a novel class of PRMT5:MEP50 inhibitors that merits further development based on the high level of target specificity.

PRMT5 has been extensively investigated in multiple human cancers. Overexpression of PRMT5 correlates with disease progression, therapeutic resistance, and poor survival. (See Xiao et al., Biomed. Pharmacother. 2019, 114, 108790.) However, few studies have evaluated the role of PRMT5 cofactors or adaptors including MEP50. The discovery that PRMT5 cooperates with pICln, but not MEP50, to activate transcription of AR and DDR genes in prostate cancer cells provides evidence that transcriptional regulation of PRMT5 target gene expression is likely dependent on the cofactors involved and potentially context-dependent. Indeed, as demonstrated by Owens et al., during the course of fractionated ionizing radiation (FIR)-induced NED, PRMT5:MEP50 mediates FIR-induced neuroendocrine differentiation (NED) and knockdown of PRMT5 significantly increases the sensitivity of LNCaP xenograft tumors to FIR, reduces tumor recurrence, and improves overall survival. (Owens et al., iScience 2020, 23 (1), 100750.) As NED is associated with therapeutic resistance and contributes to the development of neuroendocrine prostate cancer (NEPC), targeting PRMT5:MEP50 could be used to prevent treatment-induced neuroendocrine NED or even NEPC.

Because PRMT5 can repress transcription of PTEN and RBL2 in leukemia/lymphoma cell lines, targeting PRMT5:MEP50 protein-protein interaction with compounds of formula (I) may be utilized for leukemia/lymphoma treatment or as a sensitizer for other therapies by activating the PTEN/RB-family in conjunction with other disease-specific targeted therapy. As PTEN is deficient in multiple cancers and PTEN negatively regulates the PI3K-AKT-mTOR pathway, targeting PRMT5:MEP50 under specific contexts may allow indirect re-activation of PTEN and deactivation of mTOR signaling as an indirect alternative to targeting PTEN/mTOR signaling, known to be therapeutically challenging. (See Hua et al., J. Hematol. Oncol. 2019, 12 (1), 71.)

PRMT5 also plays a critical role in RNA splicing by forming a complex with MEP50 and pICln to catalyze the methylation of Sm proteins and to facilitate the assembly of spliceosome for both normal and cancer cells. (See Bezzi et al., Genes Dev. 2013, 27 (17), 1903-1916.) PRMT5 regulates splicing in both hematopoietic and neuronal stem/progenitor cells; recently, in a panel of patient-derived glioblastoma cell lines, inhibition of PRMT5-mediated alternative splicing was found to impair proliferation, induce senescence, and trigger apoptosis. Compounds of formula (I), or salts, hydrates, or solvates thereof could be used to treat multiple alternative-splicing driven diseases or progression stages (AR reactivation via AR-V7 in prostate cancer, TAK1/CD44 alternative splicing in EMT, or PTPMT1-mediated radioresistance in lung cancer). As the field continues to evolve, distinct cellular roles of PRMT5:MEP50 will continue to be uncovered, providing specific disease/context dependencies and mechanisms for patient stratification. It appears possible that targeting the PRMT5/MEP50 interaction with other compounds of formula (I) or salt, hydrates, or solvates thereof may be useful for treatment of various human diseases at different stages or processes that are dependent on the formation of the PRMT5/MEP50 complex.

RNA-seq identified significant dysregulation of TP53 signaling pathway, cellular proliferation/differentiation, and MAP Kinase signaling, each of which is a core function of PRMT5 activity in normal and cancer cells. Interestingly, only a small subset of genes was identified when compared to RNA-seq data from PRMT5 or MEP50 knockdown, suggesting a narrow scope of mechanism of action for therapeutic compounds targeting PRMT5:MEP50 protein-protein interaction. Such a narrow scope may in fact provide an added layer of specificity and selectivity for future therapeutic approaches. Further, unbiased approaches such as ChIP-seq targeting PRMT5 and MEP50 with and without compounds of formula (I) would help to identify PRMT5:MEP50-specific target genes, which may facilitate patient selection in the clinical setting.

Methylation of histone and non-histone substrates is a critical mediator of normal cell development and fate determination in differentiation as well as cancer cell proliferation and therapy resistance, necessitating clear delineation of therapeutic window and context-specific targeting strategies. PRMT5-mediated epigenetic activation/repression, alternative splicing, and PTEN/TP53 methylation, and growth factor (e.g., TGFβ/FGFR/EGFR) coactivation are all cancer cell dependencies that may be exploited via PRMT5-targeting therapies. In prostate cancer specifically, it has been demonstrated that PRMT5:MEP50 has separate and distinct roles compared to PRMT5:pICln, and it is a logical progression that more research may uncover additional cofactor-specific roles. (Owens et al., iScience 2020, 23 (1), 100750.) It is further appreciated that compounds of formula (I) provide a foundation for potent and selective therapeutic compounds.

Several illustrative embodiments of the disclosure are described by the following clauses:

A compound of the formula

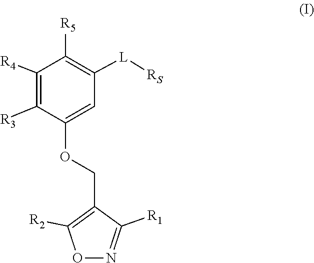

or a salt, hydrate, or solvate thereof;
wherein
   R₁ and R₂ are independently selected from the list consisting of alkyl, aryl, arylalkyl, alkenyl, cycloalkyl, heteroalkyl, heteroaryl, heteroarylalkyl, heteroalkenyl, and heterocycloalkyl, each of which is optionally substituted;
R₃, R₄ and R₅ are each independently hydrogen or —(CH₂)ₓZ^{X}, where x is an integer from 0-6 and Z^{X} is halogen, hydroxy, C₁-C₆ alkanoyloxy, optionally substituted aroyloxy, C₁-C₆ alkyl, C₁-C₆ alkoxy, C₃-C₈ cycloalkyl, C₃-C₈ cycloalkoxy, C₂-C₆ alkenyl, C₂-C₆ alkynyl, C₁-C₆ haloalkyl, C₁-C₆ haloalkoxy, C₃-C₈ halocycloalkyl, C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, nitro; —CO₂R₆, or —CONR₇R₈, where R₆, R₇, and R₈ are each independently selected in each instance from hydrogen, C₁-C₆ alkyl, aryl-C₁-C₆ alkyl, and heteroaryl-C₁-C₆ alkyl or R₇, R₈, and the nitrogen to which they are attached form an optionally substituted heterocycle;
   L is a linker;
   R_{S} is cycloalkyl, aryl or heteroaryl, each of which is optionally substituted; or R_{S} is —NR₉R₁₀, where R₉, and R₁₀ are each independently selected from hydrogen, C₁-C₆ alkyl, aryl-C₁-C₆ alkyl, and heteroaryl-C₁-C₆ alkyl; or R₉, R₁₀, and the nitrogen to which they are attached form an optionally substituted heterocycle.

In another embodiment, the compound, or a salt, a hydrate, or a solvate thereof of the preceding or any following compound clauses is described wherein the compound is not

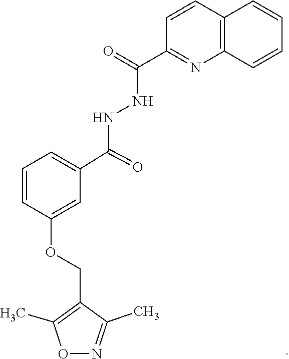 [SMILES: Cc1noc(C)c1COc1cccc(C(=O)NNC(=O)c2ccc3ccccc3n2)c1]

The compound, or a salt, a hydrate, or a solvate thereof of any one of the preceding or following clauses wherein L is selected from the group consisting of C(O)(NH)_{N}C(O), where N is 1 or 2; C(O)NHNHSO₂; C(O)NH(CH₂)_{M}NHC(O), where M is an integer from 1 to about 6; C(O)NH(CH₂)_{M2}NHSO₂, where M2 is an integer from 1 to about 6; C(O)NH(CH₂)_{M3}, where M3 is an integer from 1 to about 6; SO₂NH(CH₂)_{M4}, where M4 is an integer from 1 to about 6; and HNC(O)(CH₂)_{M4}C(O)NH, where M4 is an integer from 0 to about 4 including, for example, the compounds of Formula (Ib) identified in FIGS. 6D and 6E; and

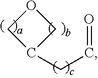

where a and b are independently 0, 1, or 2, with the proviso that a and b cannot both be 0, and c is an integer from 1 to about 4.

The compound, or a salt, a hydrate, or a solvate thereof of any one of the preceding or following clauses wherein L is selected from the group consisting of C(O)NHC(O), C(O)NHNHC(O), C(O)NHNHSO₂, C(O)NH(CH₂)₂NHC(O), C(O)NH(CH₂)₃NHC(O), C(O)NHCH₂, C(O)NH(CH₂)₂, HNC(O)C(O)NH, and

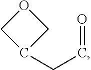

including the compounds of Formula (Ia) identified in FIG. 6C.

The compound, or a salt, a hydrate, or a solvate thereof of any one of the preceding or following compound clauses wherein one of R₃, R₄ or R₅ is halogen, amino, hydroxy, cyano, nitro, C₁-C₆ alkanoyloxy, optionally substituted aroyloxy, C₁-C₆ alkyl, C₁-C₆ alkoxy, C₃-C₈ cycloalkyl, C₃-C₈ cycloalkoxy, C₂-C₆ alkenyl, C₂-C₆ alkynyl, C₁-C₆ haloalkyl, C₁-C₆ haloalkoxy, C₁-C₆ alkylamino, (C₁-C₆ alkyl)(C₁-C₆ alkyl)amino, —CO₂R₆, or —CONR₆R₈, where R₆, R₇, and R₈ are each independently selected in each instance from hydrogen, and C₁-C₆ alkyl; and the other two of R₃, R₄ and R₅ are hydrogen.

The compound, or a salt, a hydrate, or a solvate thereof of any one of the preceding or following compound clauses wherein R₃, R₄ and R₅ are hydrogen.

The compound, or a salt, a hydrate, or a solvate thereof of any one of the preceding or following compound clauses wherein L is C(O)NHC(O).

The compound, or a salt, a hydrate, or a solvate thereof of any one of the preceding or following compound clauses wherein L is C(O)NHNHC(O).

The compound, or a salt, a hydrate, or a solvate thereof of any one of the preceding or following compound clauses wherein L is C(O)NHNHSO₂.

The compound, or a salt, a hydrate, or a solvate thereof of any one of the preceding or following compound clauses wherein L is C(O)NH(CH₂)₂NHC(O).

The compound, or a salt, a hydrate, or a solvate thereof of any one of the preceding or following compound clauses wherein L is C(O)NH(CH₂)₃NHC(O).

The compound, or a salt, a hydrate, or a solvate thereof of any one of the preceding or following compound clauses wherein L is C(O)NHCH₂.

The compound, or a salt, a hydrate, or a solvate thereof of any one of the preceding or following compound clauses wherein L is C(O)NH(CH₂)₂.

The compound, or a salt, a hydrate, or a solvate thereof of any one of the preceding or following compound clauses wherein R_{S} is optionally substituted cycloalkyl.

The compound, or a salt, a hydrate, or a solvate thereof of any one of the preceding or following compound clauses wherein R_{S} is optionally substituted aryl.

The compound, or a salt, a hydrate, or a solvate thereof of any one of the preceding or following compound clauses wherein R_{S} is —NR₉R₁₀, where R₉, and R₁₀ are each independently selected in each instance from hydrogen, C₁-C₆ alkyl, aryl-C₁-C₆ alkyl, and heteroaryl-C₁-C₆ alkyl.

The compound, or a salt, a hydrate, or a solvate thereof of any one of the preceding or following compound clauses wherein R_{S} is —NR₉R₁₀, where R₉, R₁₀, and the nitrogen to which they are attached form an optionally N-substituted aryl.

The compound, or a salt, a hydrate, or a solvate thereof of any one of the preceding or following compound clauses wherein R_{S} is optionally substituted phenyl.

The compound, or a salt, a hydrate, or a solvate thereof of any one of the preceding or following compound clauses wherein R_{S} is optionally substituted quinolinyl.

The compound, or a salt, a hydrate, or a solvate thereof of any one of the preceding or following compound clauses wherein R_{S} is optionally substituted pyridyl.

The compound, or a salt, a hydrate, or a solvate thereof of any one of the preceding or following compound clauses wherein R₁ is optionally substituted alkyl.

The compound, or a salt, a hydrate, or a solvate thereof of any one of the preceding or following compound clauses wherein R₂ is optionally substituted alkyl.

The compound, or a salt, a hydrate, or a solvate thereof of any one of the preceding or following compound clauses wherein R₁ is optionally substituted aryl.

The compound, or a salt, a hydrate, or a solvate thereof of any one of the preceding or following compound clauses wherein R₂ is optionally substituted aryl.

The compound, or a salt, a hydrate, or a solvate thereof of any one of the preceding or following compound clauses wherein R₁ is optionally substituted phenyl.

The compound, or a salt, a hydrate, or a solvate thereof of any one of the preceding or following compound clauses wherein R₂ is optionally substituted phenyl.

The compound, or a salt, a hydrate, or a solvate thereof of any one of the preceding or following compound clauses wherein R₁ is optionally substituted C₁-C₆ alkyl.

The compound, or a salt, a hydrate, or a solvate thereof of any one of the preceding or following compound clauses wherein R₂ is optionally substituted C₁-C₆ alkyl.

The compound, or a salt, a hydrate, or a solvate thereof of any one of the preceding or following compound clauses wherein R₁ is methyl.

The compound, or a salt, a hydrate, or a solvate thereof of any one of the preceding or following compound clauses wherein R₂ is ethyl.

The compound, or a salt, a hydrate, or a solvate thereof of any one of the preceding or following compound clauses wherein R₁ is methyl and R₂ is ethyl.

The compound, or a salt, a hydrate, or a solvate thereof of any one of the preceding or following compound clauses wherein R₁ and R₂ are methyl.

A pharmaceutical composition comprising one or more of the compounds, or salts, hydrates, or solvates thereof of any one of the preceding compound clauses.

A method of inhibiting protein arginine methyltransferase 5 (PRMT5) in a patient in need thereof, the method comprising the step of administering to the patient one or more of the compositions or compounds, salts, hydrates, or solvates thereof described in any of the preceding clauses.

The method of the preceding method of treatment clause wherein inhibiting the action of PRMT5 is the result of inhibiting the protein-protein interaction of PRMT5 to methylosome protein 50 (MEP50, PRMT5:MEP50 PrPrI).

The method of any one of the preceding methods clauses wherein the disease is cancer.

The method of any one of the preceding method clauses wherein the cancer is selected from the list consisting of carcinomas, sarcomas, lymphomas, Hodgkin's disease, melanomas, mesotheliomas, Burkitt's lymphoma, nasopharyngeal carcinomas, leukemias, myelomas, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The method of any of the preceding method clauses wherein the cancer is lung cancer or prostate cancer.

The method of the any of the preceding clauses wherein the cancer is prostrate cancer.

A pharmaceutical composition comprising a compound of any one of the compound clauses recited herein, and optionally comprising one or more carriers, diluents, excipients, and the like, and combinations thereof.

A pharmaceutical composition comprising a compound of any of the clauses recited herein, and optionally comprising one or more carriers, diluents, excipients, and the like, and combinations thereof for use in treating cancer in a patient.

Use of the compound, or a salt, hydrate, solvate of any of the compound clauses recited herein, or a pharmaceutical composition thereof and optionally comprising one or more carriers, diluents, excipients, and the like, and combinations thereof, in the manufacture of a medicament for treating a patient with a disease susceptible to inhibition of PRMT5:MEP50 PrPrI, such as cancer.

In reciting the foregoing collection of clauses, it is to be understood that all possible combinations of features, and all possible subgenera and sub-combinations are described. It is also to be understood that combinations of features that are chemically incompatible are excluded.

Similarly, in illustrative embodiments of the compounds of formula (I) or salts, hydrates, or solvates thereof and compositions described herein, various genera and subgenera of each of R₁, R₂, R₃, R₄, R₅, R₆, R₇, R₈, R₉, R₁₀, L, and R_{S} are described herein. It is to be understood that all possible combinations of the various genera and subgenera of each of R₁, R₂, R₃, R₄, R₅, R₆, R₇, R₈, R₉, R₁₀, L, and R_{S} are described. Each combination represents additional illustrative embodiments of compounds disclosed herein. It is to be further understood that each embodiment and each of those additional illustrative embodiments of compounds may be used in any of the compositions, methods, and/or uses described herein.

As used herein, the term “solvates” refers to compounds described herein complexed with a solvent molecule. It is appreciated that compounds described herein may form such complexes with solvents by simply mixing the compounds with a solvent or dissolving the compounds in a solvent. It is appreciated that, where the compounds are to be used as pharmaceuticals, such solvents are pharmaceutically acceptable solvents. It is further appreciated that where the compounds are to be used as pharmaceuticals. The relative amount of solvent that forms the solvate should be less than established guidelines for such pharmaceutical uses, such as less than International Conference on Harmonization (ICH) Guidelines. It is to be understood that the solvates may be isolated from excess solvent by evaporation, precipitation, and/or crystallization. In some embodiments, the solvates are amorphous, and in other embodiments, the solvates are crystalline.

In each of the foregoing and each of the following embodiments, unless otherwise indicated, it is also to be understood that the formulae include and represent any and all crystalline forms, partially crystalline forms, and non-crystalline and/or amorphous forms of the compounds, including partially ordered forms, disordered forms, liquid crystal forms, and meso phases of any of the foregoing.

In each of the foregoing and each of the following embodiments, unless otherwise indicated, it is also to be understood that the formulae include and represent each possible isomer, such as stereoisomers and geometric isomers, both individually and in any and all possible mixtures. Furthermore, one or more hydrogen atoms can be replaced with deuterium. As deuterium and hydrogen have nearly the same physical properties, deuterium substitution is the smallest structural change that can be made. Deuteration is well-known to those of ordinary skill in the art.

In each of the foregoing and each of the following embodiments, unless otherwise indicated, it is also to be understood that the transitional phrase “consisting essentially of” means that the scope of the corresponding composition, unit dose, method or use is understood to encompass the specified compounds or recited steps, and those that do not materially affect the basic and novel characteristics of the composition described herein. For example, a method described herein that consists essentially of a single compound, or genus of compounds, is understood to represent a monotherapy for the recited disease. Though the monotherapy may include co-administration of one or more carriers, vehicles, diluents, adjuvants, excipients, and the like, and combinations thereof, and/or include co-administration of one or more additional active pharmaceutical ingredients, those latter additional active pharmaceutical ingredients are to be understood to be for treating diseases and/or symptoms distinct from treating the underlying conditions described herein.

It is to be understood that each of the foregoing embodiments may be combined in chemically relevant ways to generate subsets of the embodiments described herein. Accordingly, it is to be further understood that all such subsets are also illustrative embodiments of the disclosure described herein.

The compounds described herein may contain one or more chiral centers or may otherwise be capable of existing as multiple stereoisomers. It is to be understood that in one embodiment, the compounds described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E. and Z double bonds, or spatial arrangements, such as cis, trans, syn, and anti, relative configurations on a ring. It is to be understood that in another embodiment, the disclosure is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₉, C₂-C₆, and C₂-C₄, and C₃-C₈, C₃-C₆, and C₃-C₄, and the like may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments disclosed herein, it is to be understood, in each case, that the recitation of alkyl refers to alkyl as defined herein, and optionally lower alkyl. In embodiments of the disclosure, it is to be understood, in each case, that the recitation of alkenyl refers to alkenyl as defined herein, and optionally lower alkenyl. In embodiments of the disclosure, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₅, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like. Illustratively, such particularly limited length alkenylene and/or alkynylene groups, including C₂-C₈, C₂-C₆, and C₂-C₄, and C₃-C₈, C₃-C₆, and C₃-C₄, and the like may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the disclosure, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamiino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonvl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

As used herein, the term “sulfinic acid or a derivative thereof” includes SO₂H and salts thereof, and esters and amides thereof.

As used herein, the term “sulfonic acid or a derivative thereof” includes SO₃H and salts thereof, and esters and amides thereof.

As used herein, the term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

As used herein, the term “phosphinic acid or a derivative thereof” includes P(R)O₂H and salts thereof, and esters and amides thereof, where R is alkyl, alkenyl, alkynyl, cycloalkyl, cycloalkenyl, heteroalkyl, heteroalkenyl, cycloheteroalkyl, cycloheteroalkenyl, aryl, heteroaryl, arylalkyl, or heteroarylalkyl, each of which is optionally substituted.

As used herein, the term “phosphonic acid or a derivative thereof” includes PO₃H₂ and salts thereof, and esters and amides thereof.

As used herein, the term “hydroxylamino and derivatives thereof” includes NHOH, and alkyloxylNH, alkenyloxylNH, alkynyloxylNH, heteroalkyloxylNH, heteroalkenyloxylNH, heteroalkynyloxylNH, cycloalkyloxylNH, cycloalkenyloxylNH, cycloheteroalkyloxylNH, cycloheteroalkenyloxylNH, aryloxvlNH, arylalkyloxylNH, arylalkenyloxylNH, arylalkynyloxylNH, heteroaryloxylNH, heteroarylalkyloxylNH, heteroarylalkenyloxylNH, heteroarylalkynyloxylNH, acyloxyNH, and the like, each of which is optionally substituted.

As used herein, the term “hydrazino and derivatives thereof” includes HNNH, alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHINH, arylalkylNHNH, arylalkenylNHNH, arylalkynylNHNH, heteroarylNHNH, heteroarylalkylNHlNH, heteroarylalkenylNHNH, heteroarylalkynylNHNH, acylNHNH, and the like, each of which is optionally substituted.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents or heteroaryl substituents, respectively, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative optional substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R₄ and —CONR₅R₆, where R₄, R₅, and R₆ are each independently selected in each occurrence from hydrogen, C₁-C₆ alkyl, aryl-C₁-C₆ alkyl, and heteroaryl-C₁-C₆ alkyl.

As used herein, the term “linker” generally refers to a chain of atoms that covalently connects R_{S} to the remainder of compounds of formula (I). Illustratively, the chain of atoms is selected from C, N, O, S, Si, and P, or C, N, O, S, and P, or C, N, O, and S. T. The linker may have a wide variety of lengths, such as in the range from about 2 to about 15 atoms in the contiguous backbone. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxylamines, or when combined with carbonyl groups forming urethanes, amino acids, acyloxylamines, hydroxamic acids, and the like; and many others. In addition, it is to be understood that the atoms forming the chain in each of the foregoing illustrative embodiments may be either saturated or unsaturated, thus forming single, double, or triple bonds, such that for example, alkanes, alkenes, alkynes, imines, and the like may be radicals that are included in the linker. In addition, it is to be understood that the atoms forming the linker may also be cyclized upon each other or be part of cyclic structure to form divalent cyclic structures that form the linker, including cycloalkanes, cyclic ethers, cyclic amines, and other heterocycles, arylenes, heteroarylenes, and the like in the linker. In this latter arrangement, it is to be understood that the linker length may be defined by any pathway through the one or more cyclic structures. Illustratively, the linker length is defined by the shortest pathway through the each one of the cyclic structures. It is to be understood that the linkers may be optionally substituted at any one or more of the open valences along the chain of atoms, such as optional substituents on any of the carbon, nitrogen, silicon, or phosphorus atoms.

The compounds described herein can be used for both human clinical medicine and veterinary applications. Thus, the patient treated with the compounds described herein can be human or, in the case of veterinary applications, can be a laboratory, agricultural, domestic, or wild animal. The present disclosure can be applied to patients including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The compounds, compositions, methods, uses, kits, and unit doses disclosed herein can be used to treat cancer. Illustrative examples of cancers that can be treated are carcinomas, sarcomas, lymphomas, Hodgekin's disease, melanomas, mesotheliomas, Burkitt's lymphoma, nasopharyngeal carcinomas, leukemias, and myelomas. Illustrative cancers include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, lung cancers, and the like.

It is to be understood that in every instance disclosed herein, the recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

It is also to be understood that unless otherwise indicated the recitation of a numerical value necessarily reflects the relative precision of the numerical value. For example, the recitation of a number with a specified precision based on significant figures necessarily includes a range of values that would match that number after appropriate rounding. For example, the recitation of the number 1 with a single significant figure is understood to properly refer to a range of values from 0.5 to 1.4. Similarly, the recitation of the number 1.0 with two significant figures is understood to properly refer to a range of values from 0.95 to 1.04. The relative precision of the numerical value can be further indicated by modifying with the term “about” to indicate that the modified number has lower precision.

As used herein, the term “composition” generally refers to any product comprising the indicated ingredients in the listed amounts, as well as any product which results, directly or indirectly, from combinations of the specified ingredients in the specified amounts. It is appreciated that certain functional groups, such as the hydroxy, amino, and like groups form complexes and/or coordination compounds with water and/or various solvents, in the various physical forms of the compounds. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. In addition, it is to be understood that the compositions may be prepared from various co-crystals of the compounds described herein. Accordingly, such pharmaceutical compositions that recite compounds described herein are to be understood to include each of, or any combination of, the various morphological forms and/or solvate or hydrate forms of the compounds described herein.

Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington, The Science and Practice of Pharmacy, 23rd Edition, 2020)).

As used herein, the term “patient” generally refers to mammals, including humans, companion animals, and livestock animals. A patient in need of relief is a patient who has or is suffering from a disease described herein.

As used herein, the term “inhibiting” when referencing treatment of a patient generally includes its generally accepted meaning which includes prohibiting, preventing, restraining, slowing, stopping, and/or reversing progression, severity of the disease and/or any resultant symptom of the disease. As such, the methods described herein include both clinical therapeutic and/or prophylactic administration, as appropriate.

As used herein, the term “inhibiting” when used in the context of a biochemical or biological interaction includes its generally accepted meaning which includes, preventing, restraining, slowing, or stopping the interaction.

The term “therapeutically effective amount” as used herein, refers to that amount of active compound or pharmaceutical agent that elicits the biological or medicinal response in a tissue system, animal or human that is being sought by a researcher, veterinarian, medical doctor, or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and/or vehicles.

As used herein, the term “carrier” generally refers to any ingredient other than the active components in a formulation. The choice of carrier will to a large extent depend on factors such as the particular mode of administration, the effect of the carrier on solubility and stability, and the nature of the dosage form.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustrative means of parenteral administration include needle (including microneedle) injectors, needle-free injectors, and infusion techniques, as well as any other means of parenteral administration recognized in the art. Parenteral formulations are typically aqueous solutions which may contain excipients such as salts, carbohydrates, and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art.

Illustratively, administering includes local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the patient being treated. It is further appreciated that local administration may be directly in the injury site, or locally in the surrounding tissue. Similar variations regarding local delivery to particular tissue types, such as organs, and the like, are also described herein. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

Illustrative examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions can be formulated so as to provide quick, sustained, or delayed release of the active ingredient after administration to the patient by employing procedures known in the art. It is to be understood that one or more carriers, one or more diluents, one or more excipients, and combinations of the foregoing may be used in making the pharmaceutical compositions described herein. It is appreciated that the carriers, diluents, and excipients used to prepare the compositions described herein are advantageously GRAS (generally regarded as safe) compounds. It is also appreciated that acids and bases used to make salts, as described herein, and/or solvents used to make solvates, as described herein, are also advantageously GRAS compounds.

Illustrative examples of emulsifying agents include naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE. Examples of chelating agents are sodium EDTA, citric acid, and phosphoric acid. Examples of gel forming agents are CARBOPOL, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN)).

It is to be understood that therapeutically effect doses administered in animal models may be used to calculate corresponding therapeutically effect doses for administration to other patients, including humans. Illustrative corresponding doses may be calculated using the Office of New Drugs in the Center for Drug Evaluation and Research's (CDER) Guidance for Industry Estimating the Maximum Safe Starting Dose in Initial Clinical Trials for Therapeutics in Adult Healthy Volunteers, July 2005, and which is incorporated herein in its entirety by reference.

The following examples further illustrate specific embodiments of the invention; however, the following illustrative examples should not be interpreted in any way to limit the invention. Unless otherwise indicated, all starting compounds, reagents, and solvents used in the following examples are available from commercial suppliers.

### Examples

### Methods

### Computational Modeling, Docking, And Binding Energy Prediction

Virtual screening was conducted with ZINCPharmer. Refinement was performed (list of compounds was minimized) using SMINA. Binding energies were predicted via web server for FastContact.

### Cell Lines and Cell Culture.

LNCaP, COS-1, and A549 cell lines were purchased from ATCC. Routine mycoplasma screening was performed using the LookOut PCR Mycoplasma Detection Kit (Sigma), as described in Owens et al., iScience 2020, 23 (1), 100750, which is incorporated herein by reference in its entirety. Cells were stored as frozen stock in vapor phase of LN₂ and thawed prior to use. Cell lines were cultured 3 passages after thawing prior to experimentation and maintained for no longer than 30 total passages. LNCaP cells were cultured in RPMI 1640 (Corning), and COS-1 cells were cultured in DMEM (Corning) medium. A549 cells were cultured in Hink's F12K Medium (Corning). All media were supplemented with 10% FBS (Atlanta Biologicals), 1 mM sodium pyruvate (Corning), penicillin (100 units/mL) and streptomycin (100 μg/mL) combination (Gibco), and 2 mM/L L-glutamine (Corning). Knockdown cell lines were generated using the pLKO-Tet-On system. The pLKO-Tet-On plasmid for shRNA expression was obtained from Addgene, as described in Wiederschain et al. Cell Cycle 2009, 8 (3), 498-504, which is incorporated herein by reference in its entirety, shRNA read frames that target PRMT5 and MEP50 were utilized for stable cell line generation, as described in Deng et al. Oncogene 2017, 36 (9), 1223-1231, which is incorporated herein by reference in its entirety.

For dox-induced PRMT5, MEP50, or scrambled control knockdown cell lines, doxycycline was applied at the final concentration of 1 μg/mL every 48 hours to establish and maintain PRMT5 knockdown (shPRMT5), MEP50 knockdown (shMEP50), or express scramble control shRNA (shSC). Cells were harvested in Trizol and RNA was purified for RNAseq (Ambion) following methodology described in Owens et al., Mol. Cancer Ther. 2022, MCT-21-0103-A.2021, which is incorporated herein by reference in its entirety.

### Bifc Assay and Screening

BiFC Mutation Assay: COS-1 cells were grown in DMEM and seeded to 100,000 cells/well of a 12-well plate and allowed to attach for 24 hours. Cells were transfected with 400 ng/well of pMyc-VN-PRMT5 (WT or mutant) BiFC plasmid, 400 ng/well pHA-VC-MEP50 (WT or mutant) BiFC plasmid, and 200 ng/well pHA-Cerulean transfection control. Following 18 hours after transfection, the cells were imaged on a Nikon TE-2000U microscope and images for CFP, YFP, and phase contrast were acquired using MetaMorph software (Nikon) with 10× objective. Images were analyzed with ImageJ, as described in Schneider et al., Nat. Methods 2012, 9 (7), 671-675, which is incorporated herein by reference in its entirety. Regions of Interest (ROI) were selected around each cell, and mean intensity was measured for each selection. A YFP:CFP ratio was calculated for DMSO as well as control treatment cells. The YFP:CFP ratio was then normalized to that of DMSO to generate the BiFC Efficiency score. All BiFC mutant experiments are performed as three biological replicates. To ensure comparable expression of BiFC plasmids, cells were subsequently washed with PBS and harvested in 100 μL of 2×SDS sample buffer and analyzed via western blot. Anti-HA antibody was used to detect MEP50 fusions and Cerulean expression. Anti-Myc antibody was used to detect PRMT5 fusions.

BiFC Drug Screens: COS-1 cells were grown in DMEM and seeded to 50,000 cells/well of a 12-well plate and allowed to attach for 24 hours. Cells were then transfected with three plasmids pMyc-VN155-PRMT5, pHA-VC-MEP50, and pFLAG-NLS-CFP to visualize the interaction between PRMT5:MEP50. For BiFC screens, the COS-1 cells were treated with compound or DMSO to final concentration (10 μM for compound 1-12 screen and 0.25, 0.50, 0.75, 1.0, and 5.0 μM or subsequent compound 8 screen) six hours after transfection and returned to the incubator. Following 24 hours after transfection, cells were imaged on a Nikon TE-2000U microscope and images for CFP, YFP, and phase contrast were acquired using MetaMorph software (Nikon) with 20× objective. Images were analyzed with ImageJ⁷⁰. Regions of Interest (ROI) were selected around each cell, and mean intensity was measured for each selection. A YFP:CFP ratio was calculated for DMSO as well as control treatment cells. The YFP:CFP ratio was then normalized to that of DMSO to generate the BiFC Efficiency score. The lower score indicated less PRMT5:MEP50 PPI detected in a given cell or treatment group. Inhibition (% Inhibition) is calculated as a percent of 100%−the BiFC Efficiency. Referring to FIGS. 6A-6C, for the BiFC screen of compounds 13-22 (including 8a and 8b), COS-1 cells were transfected for 24 hours, treated with 0.5 μM of compounds for 18 hours, and then imaged as described above. Referring to FIG. 6C, for the BiFC screen of compounds 23-26 (including 8a and 8b), COS-1 cells were transfected for 24 hours, treated with 0.25 M of compounds for 18 hours, and then imaged as described above. BiFC drug screens were performed in single biological replicate as a high throughput screen to produce ranked order of compounds, although multiple biological replicate data was used whenever available.

### MTT Assay

LNCaP cells were seeded at 7,000 cells per well of a 96-well cell culture plate and incubated 24 hours to allow for attachment. Test compounds were diluted in RPMI-1640/25% DMSO pre-dilutions and added to respective wells of the assay plate to maintain constant concentration of 0.25% DMSO. After addition of compounds, cells were returned to incubator (37° C., 5% CO₂) for 72 hours. Following incubation, assay plates were removed from incubator and media aspirated. 30 μL complete RPMI supplemented with 0.5 mg/mL MTT (Sigma) was added to the plate, and plate returned to incubator for 4 hours. Plates were removed, and 88 μL DMSO was added. Plates were shaken at 700 rpm for 1 minute and read on spectrophotometer at 570 nm.

### RT-QPCR Assay

LNCaP cells were seeded to either 6 cm or 10 cm dishes at 800,000 or 2,200,000 cells/dish respectively. Cells were allowed to attach for 24 hours and then subsequently treated with either compound 17 (500 nM) or DMSO for 72 hours. Cells were then harvested with Trizol reagent (Ambion) and RNA integrity was verified via agarose gel electrophoresis. Promega High Capacity cDNA Reverse Transcription Kit (Promega) was utilized following manufacturer instructions and as described previously, as described in Deng et al., Oncogene 2017, 36 (9), 1223-123; Hsu et al., Mol. Cell. Endocrinol. 2013, 372 (1-2), 12-221; and Zhang et al., Biochim. Biophys. Acta 2014, 1839 (11), 1330-1340, each of which is incorporated herein by reference in its entirety for its teachings regarding same. RT-qPCR was performed with FastStart Universal SYBR Green Master Mix (Thermo Fisher Scientific) and detected on a QuantStudio 6 Flex with QuantStudio Real-Time PCR control software (Thermo Fisher Scientific). QuantStudio Design and Analysis software (Thermo Fisher Scientific) was used for data analysis. Technical triplicates were run for all samples, samples without detectable amplification were deemed undetected. Primer sets were validated via melt curve and agarose gel analysis of RT-qPCR product. AR primers were used as described in Deng et al., Oncogene 2017, 36 (9), 1223-123. IVL primers were used as described in Saha et al., J. Invest. Dermatol. 2016, 136 (1), 214-224, which is incorporated herein by reference in its entirety for its teachings regarding same.

### Co-Immunoprecipitation

LNCaP cells were treated with either DMSO or compound 17 (500 nM, 24 hrs) and harvested for Co-IP and WB in lysis buffer composed of 50 mM Tris-HCl, pH 7.4, 100 mM NaCl, 10 mM EDTA, 0.1% Triton X-100, 1 mM DTT, 1 mM PMSF, and 5 μg each of chymostatin, leupeptin, pepstatin A, and antipan. Cells were lysed over 1 hour rotating in 4° C. and supernatant cleared. Total lysate (1 mg/mL) was used for Co-IP with 4 μg rabbit anti-PRMT5 pAb (Millipore Sigma 07-405), rabbit anti-MEP50 pAb (Cell Signaling Technologies S2823S) or normal rabbit IgG (Millipore Sigma N01-100 UG) overnight. Antibody-bound proteins were precipitated with Pierce Protein A agarose beads (Thermo Scientific 20333). Antibodies and immunoprecipitated proteins were prepared for western blot by adding 50 μL 2×SDS buffer, boiling at 95° C. for 5 min, and storing at −80° C. or proceeding to western blot.

### Western Blot Assay

Co-IP product, input sample, or ladder were loaded into a 10% acrylamide/bisacrylamide gel (20 μL Co-IP, 20 μL input (0.4% total), 5 μL ladder per lane). Gel was run 90 min @125 V and transferred onto a nitrocellulose membrane for 75 min at 100 V. The membrane was washed and incubated with either anti-PRMT5 rabbit pAb (1:1000 in phosphate buffered saline, pH 7.4, supplemented with Tween-20 (PBST), Millipore 07-405) or anti-MEP50 mouse mAb (1:1000 in PBST, Invitrogen MA5-32970). Secondary anti-rabbit IgG-HRP conjugate (1:1000 in PBST, GE Healthcare) or anti-mouse IgG-HRP conjugate (1:1000 in PBST, GE Healthcare) was used to provide signal for the blot which was subsequently imaged on a Bio-Rad ChemiDoc Touch Imaging System (Bio-Rad). Band Intensity was determined with ImageLab software and ImageJ.

For global histone H4R3 and H4R3me2s western blots, cell lysate was prepared in RIPA buffer (10 mM Tris-HCl pH 8.0, 5 mM EDTA, 1% Triton X-100, 0.1% sodium deoxycholate, 0.1% sodium dodecyl sulfate, 150 mM sodium chloride, and 5 μg/mL each chymostatin, leupeptin, pepstatin A, and antipan in DMSO, with 1 mM PMSF and total soluble protein was quantified using Bradford assay. 100 μL lysate was combined with 100 μL 2×SDS buffer, and 20 μg total lysate was loaded into the wells of a 15% acrylamide/bisacrylamide gel. The gel was run for 60 min at 125 V and transferred onto nitrocellulose membrane for 45 min at 100 V. Anti-H4R3 rabbit pAb (Abcam, ab10158) or anti-H4R3me2s rabbit pAb (Abcam, ab5823) was diluted 1:1000 in PBST and incubated overnight at 4° C. Secondary IRDyeR 800CW Donkey anti-Rabbit IgG Secondary Antibody (Li-Cor, 926-32213) was diluted 1:20,000 in PBST and incubated with the membrane, which was then imaged via LiCor Odyssey CLx imager and analyzed with ImageStudioLite software (Li-Cor). Integrated intensity of H4R3me2s band was normalized to H4R3 to determine relative abundance of H4R3me2s across DMSO and Cpd 17-treated samples.

For confirmation of BiFC plasmid expression, 100 μL 2×SDS buffer was loaded into the wells of the 24-well plate used for the BiFC screen and harvested. To the lanes of a 10% SDS-PAGE gel, 20 μL lysate was loaded. The gel was run for 60 min at 125 V and transferred onto nitrocellulose membrane for 75 min at 100 V. The membrane was incubated with either anti-HA tag antibody (Sigma-Aldrich, H3663) for detection of HA-fusion Cerulean protein and HA-fused MEP50 (wild type or mutants) or anti-Myc tag antibody (Abcam, Clone 9E10, ab32) for detection of Myc-fusion PRMT5 (wild type or mutants) at 1:1,000 dilution in PBST. Secondary antibody IRDye®800CW Donkey anti-Mouse IgG (LI-COR, 926-32212) was used for detection, and membranes were read on LI-COR Odyssey imager.

### RNA Sequencing Analysis

The reads were mapped to the human genome hg38 using STAR (v2.7.2a) as described in Dobin et al. Bioinforma. Oxf Engl. 2013, 29 (1), 15-21, which is incorporated herein by reference in its entirety. RNA-seq aligner was implemented using the following parameter: “--outSAMmapqUnique 60”. Uniquely mapped sequencing reads were assigned to GENCODE 31 gene using featureCounts (v2.0.1) (Liao et al., Bioinforma. Oxf. Engl. 2014, 30 (7), 923-930, which is incorporated herein by reference in its entirety) with the following parameters: “-p-Q 10-O”. The data was filtered using read count >10 in at least 3 of the samples, normalized using TMM (trimmed mean of M values) method and subjected to differential expression analysis using edgeR (v3.34.1), as described in Robinson et al., Bioinforma. Oxf. Engl. 2010, 26 (1), 139-140, and McCarthy et al., Nucleic Acids Res. 2012, 40 (10), 4288-4297, each of which is incorporated by reference in its entirety. Gene ontology and KEGG pathway functional analysis was performed on differential expression gene with p value cut-off of 0.05 using DAVID, as set forth in Dennis et al., Genome Biol. 2003, 4 (5), P3, and Huang et al., Nat. Protoc. 2009, 4 (1), 44-57, each of which is incorporated by reference in its entirety.

### Chemical Synthesis—General Methods

NMR spectra were recorded on Bruker spectrometers (¹H at 400 MHz, 500 MHz, 800 MHz and ¹³C at 100 MHz, 125 MHz, 200 MHz). Chemical shifts (6) were given in ppm with reference to solvent signals [¹H NMR: CHCl₃ (7.26); ¹³C NMR: CDCl₃ (77.2), C₆D₆ (128.02), CD₃OD (49.0)]. Column chromatography was performed on silica gel. All reactions sensitive to air or moisture were conducted under argon atmosphere in dry and freshly distilled solvents under anhydrous conditions, unless otherwise noted. Anhydrous THF and toluene were distilled over sodium benzophenone ketyl under Argon. Anhydrous CH₂Cl₂ was distilled over calcium hydride under Argon. All other solvents and reagents were used as obtained from commercial sources without further purification. All compounds tested in the biological assays are >95% purity based on NMR analysis or HPLC analysis.

(3,5-dimethylisoxazol-4-yl)methanol (28): To a 0° C. solution of 3,5-dimethyl-1,2-oxazole-4-carbaldehyde (1.0 g, 8.0 mmol) in anhydrous methanol (60 mL) was added sodium borohydride (450 mg, 12.0 mmol). The reaction mixture was stirred at room temperature overnight. Methanol was evaporated and water (50 mL) was added. The resultant mixture was extracted with EtOAc (3×50 mL). The organic extracts were combined, dried over Na₂SO₄, filtered, evaporated, and subjected to the flash column chromatography to afford 28 (817 mg, 80% yield) as white solid. ¹H NMR (500 MHz, CDCl₃) δ=4.38 (s, 2H), 2.32 (s, 3H), 2.21 (s, 3H); ¹³C NMR (125 MHz, CDCl₃) δ=166.7, 159.8, 113.8, 53.4, 10.9, 9.9. MS (ESI): m/z 128.1 calc. for C₆H₁₀NO₂⁺ [M+H]⁺, found 128.2.

Methyl 3-((3,5-dimethylisoxazol-4-yl)methoxy)benzoate (31): To a solution of 28 (540 mg, 4.2 mmol) in anhydrous CH₂Cl₂ (42 mL) was added dropwise phosphorus tribromide (1.3 mL, 12.7 mmol). The mixture was stirred at room temperature for 3 hours. Water (50 mL) was added. The resultant mixture was extracted with CH₂Cl₂ (3×50 mL). The organic extracts were combined, dried over MgSO₄, filtered, evaporated, and the residue was dried in vacuo, affording crude 29 for the next step without further purification.

To a stirred solution of 3-phenolic methyl ester 30 (571 mg, 3.8 mmol) in DMF (26 mL) at room temperature, was added potassium carbonate (1 g, 7.5 mmol) followed by 29 (710 mg, 3.8 mmol). The reaction mixture was stirred overnight. The mixture was filtered over a celite pad and washed with EtOAc (5×60 mL). The organic extracts were combined, dried over MgSO₄, filtered, and evaporated. The crude residue was purified by flash column chromatography to afford 31 (900 mg, 93% yield). ¹H NMR (500 MHz, CDCl₃) δ 7.68 (dt, J=7.8, 1.2 Hz, 1H), 7.61 (dd, J=2.7, 1.5 Hz, 1H), 7.36 (t, J=7.9 Hz, 1H), 7.12 (ddd, J=8.2, 2.7, 1.0 Hz, 1H), 4.84 (s, 2H), 3.92 (s, 3H), 2.42 (s, 3H), 2.30 (s, 3H); ¹³C NMR (125 MHz, CDCl₃) δ=167.7, 166.8, 159.8, 158.2, 131.6, 129.6, 122.7, 120.5, 114.6, 110.0, 59.7, 52.3, 11.2, 10.2. MS (ESI): m/z 262.1 calc. for C₁₄H₁₆NO₄⁺ [M+H]⁺, found 262.4.

3-((3,5-dimethylisoxazol-4-yl)methoxy)benzohydrazide (32): A solution of hydrazine hydrate (80%, 2.09 mL, 35 mmol) was added dropwise to a solution of 31 (2.3 mmol) in EtOH (12 mL). The reaction mixture was refluxed for 12 hours until completion. After cooling, water (10 mL) was added, and the precipitate was filtered and washed with a small amount of ethanol and water. The crude product was subjected to the next step without further purification.

A general procedure for the synthesis of compounds 8b, 13-20: Acyl chloride (0.12 mmol) in CH₂Cl₂ (1.0 mL) was added dropwise to a dried round flask containing the corresponding benzohydrazide (0.11 mmol), pyridine (44 μL, 0.55 mmol), and DMAP (1.5 mg, 0.01 mmol) in CH₂Cl₂ (1.0 mL) at 0° C. The mixture was stirred at room temperature for 12 hours and then washed with dilute aqueous HCl and water and dried over Na₂SO₄. After removal of the solvent at reduced pressure, the crude product was purified by flash column chromatography to obtain the desired product.

N′-(3-((3,5-dimethylisoxazol-4-yl)methoxy)benzoyl)quinoline-2-carbohydrazide (8b, 46%): ¹H NMR (500 MHz, CDCl₃) δ 10.70 (s, 1H), 10.07 (s, 1H), 8.31-8.25 (m, 1H), 8.19 (d, J=8.4 Hz, 1H), 8.12 (dd, J=8.4, 1.2 Hz, 1H), 7.86 (dd, J=8.2, 1.4 Hz, 1H), 7.78 (ddd, J=8.4, 6.9, 1.4 Hz, 1H), 7.64 (ddd, J=8.1, 6.8, 1.2 Hz, 1H), 7.59-7.48 (m, 2H), 7.31 (t, J=7.9 Hz, 1H), 7.05 (ddd, J=8.3, 2.6, 0.9 Hz, 1H), 4.77 (s, 2H), 2.36 (s, 3H), 2.25 (s, 3H); ¹C NMR (125 MHz, CDCl₃) δ 167.7, 164.5, 162.0, 159.8, 158.6, 147.8, 146.6, 137.6, 132.9, 130.5, 130.0, 129.9, 129.6, 128.5, 127.7, 120.2, 119.9, 118.6, 112.6, 110.0, 59.6, 11.1, 10.1. HRMS (ESI): m/z 417.1557 calc. for C₂₃H₂₁N₄O₄⁺ [M+H]⁺, found 417.1561.

N′-(3-((3,5-dimethylisoxazol-4-yl)methoxy)benzoyl)quinoline-8-sulfonohydrazide (13, 77%): ¹H NMR (500 MHz, CDCl₃) δ 9.39 (s, 1H), 9.18 (dd, J=4.3, 1.7 Hz, 1H), 8.39 (dd, J=7.3, 1.4 Hz, 1H), 8.30 (dd, J=8.3, 1.8 Hz, 2H), 8.09 (dd, J=8.2, 1.4 Hz, 1H), 7.65-7.58 (m, 2H), 7.30 (t, J=7.9 Hz, 1H), 7.20 (d, J=7.7 Hz, 1H), 7.15 (s, 1H), 7.04 (ddd, J=8.3, 2.7, 1.0 Hz, 1H), 4.72 (s, 2H), 2.35 (s, 3H), 2.23 (s, 3H); ¹³C NMR (125 MHz, CDCl₃) δ 167.6, 165.0, 159.6, 158.6, 151.3, 143.8, 136.9, 136.0, 134.2, 132.6, 131.2, 130.0, 128.8, 125.3, 122.6, 119.7, 119.6, 113.0, 109.8, 59.6, 11.1, 10.1. HRMS (ESI): m/z 453.1227 calc. for C₂₂H₂₁N₄O₅S⁺ [M+H]⁺, found 453.1222.

N′-(3-((3,5-dimethylisoxazol-4-yl)methoxy)benzoyl)-2-naphthohydrazide (14, 47%): ¹H NMR (500 MHz, CDCl₃) δ 9.64 (q, J=6.1 Hz, 2H), 8.42 (d, J=1.4 Hz, 1H), 7.93-7.86 (m, 4H), 7.58 (m, 2H), 7.52-7.47 (m, 2H), 7.37 (t, J=7.9 Hz, 1H), 7.13-7.09 (m, 1H), 4.80 (s, 2H), 2.40 (s, 3H), 2.28 (s, 3H); ¹³C NMR (125 MHz, CDCl₃) δ 167.7, 164.4, 163.9, 159.7, 158.7, 135.2, 132.8, 132.5, 130.1, 129.1, 128.8, 128.3, 127.8, 127.1, 123.2, 119.8, 119.8, 112.9, 109.9, 59.7, 11.2, 10.2. HRMS (ESI): m/z 416.1605 calc. for C₂₄H₂₂N₃O₄⁺ [M+H]⁺, found 416.1610.

N′-(3-((3,5-dimethylisoxazol-4-yl)methoxy)benzoyl)-5-(trifluoromethyl)picolinohydrazide (15, 96%): ¹H NMR (500 MHz, CDCl₃) δ 10.54 (d, J=5.2 Hz, 1H), 9.72 (d, J=5.2 Hz, 1H), 8.85 (dd, J=1.5, 0.8 Hz, 1H), 8.24 (dt, J=8.2, 0.8 Hz, 1H), 8.13-8.07 (m, 1H), 7.53-7.47 (m, 2H), 7.33 (t, J=7.9 Hz, 1H), 7.08 (ddd, J=8.3, 2.6, 0.9 Hz, 1H), 4.80 (s, 2H), 2.40 (s, 3H), 2.27 (s, 3H); ¹³C NMR (125 MHz, CDCl₃) δ 167.7, 164.4, 160.1, 159.7, 158.6, 151.0, 145.7, 145.7, 134.9, 132.7, 130.0, 129.5 (q, J=33.75 Hz), 122.9 (q, J=271.25 Hz), 122.3, 119.9 (d, J=11.25 Hz), 112.9, 109.9, 59.6, 11.1, 10.1. ¹⁹F NMR (470 MHz, CDCl₃) δ −63.8. HRMS (ESI): m/z 457.1094 calc. for C₂₀H₁₇F₃N₄NaO₄¹ [M+Na]¹, found 457.1101.

N′-(3-((3,5-dimethylisoxazol-4-yl)methoxy)benzoyl)-6-(trifluoromethyl)picolinohydrazide (16, 99%): ¹H NMR (500 MHz, CDCl₃) δ 10.26 (d, J=4.9 Hz, 1H), 9.10 (d, J=4.9 Hz, 1H), 8.40-8.35 (m, 1H), 8.11 (td, J=7.9, 0.7 Hz, 1H), 7.89 (dd, J=7.9, 1.0 Hz, 1H), 7.53-7.46 (m, 2H), 7.39 (t, J=7.9 Hz, 1H), 7.12 (ddd, J=8.2, 2.6, 1.0 Hz, 1H), 4.84 (s, 2H), 2.43 (s, 3H), 2.30 (s, 3H); ¹³C NMR (125 MHz, CDCl₃) δ 167.8, 164.2, 159.8, 159.7, 158.7, 148.4, 147.5 (d, J=36.25 Hz), 139.4, 132.9, 130.1, 125.3, 123.6, 119.9, 119.7, 113.1, 109.9, 59.7, 11.2, 10.2; ¹⁹F NMR (470 MHz, CDCl₃) δ −69.0. HRMS (ESI): m/z 435.1275 calc. for C₂₀H₁₈F₃N₄O₄⁺ [M+H]⁺, found 435.1281.

N′-(3-((3-ethyl-5-methylisoxazol-4-yl)methoxy)benzoyl)quinoline-2-carbohydrazide (17, 39%): ¹H NMR (500 MHz, CDCl₃) δ 10.51 (s, br, 1H), 9.64 (s, br, 1H), 8.33 (d, J=8.5 Hz, 1H), 8.24 (d, J=8.4 Hz, 1H), 8.17 (dd, J=8.5, 1.1 Hz, 1H), 7.92-7.88 (m, 1H), 7.81 (ddd, J=8.4, 6.9, 1.5 Hz, 1H), 7.66 (ddd, J=8.1, 6.8, 1.2 Hz, 1H), 7.57-7.54 (m, 1H), 7.51 (dt, J=7.7, 1.2 Hz, 1H), 7.38 (t, J=7.9 Hz, 1H), 7.11 (ddd, J=8.3, 2.6, 0.9 Hz, 1H), 4.83 (s, 2H), 2.69 (q, J=7.6 Hz, 2H), 2.41 (s, 3H), 1.29 (t, J=7.5 Hz, 3H); ¹³C NMR (125 MHz, CDCl₃) δ 167.8, 164.4, 164.0, 161.3, 158.7, 147.7, 146.7, 137.7, 133.1, 130.6, 130.0, 130.0, 129.6, 128.5, 127.8, 119.9, 119.8, 118.7, 112.8, 109.3, 59.6, 18.7, 12.2, 11.2. HRMS (ESI): m/z 453.1533 calc. for C₂₄H₂₂N₄NaO₄⁺ [M+Na]⁺, found 453.1539.

N′-(3-((5-ethyl-3-methylisoxazol-4-yl)methoxy)benzoyl)quinoline-2-carbohydrazide (18, 71%): ¹H NMR (500 MHz, CDCl₃) δ 10.70 (s, 1H), 9.53 (s, 1H), 8.33 (d, J=8.4 Hz, 1H), 8.23 (d, J=8.4 Hz, 1H), 8.16 (dd, J=8.5, 1.2 Hz, 1H), 7.89 (dd, J=8.3, 1.4 Hz, 1H), 7.80 (ddd, J=8.4, 6.9, 1.4 Hz, 1H), 7.66 (ddd, J=8.1, 6.9, 1.2 Hz, 1H), 7.56 (dd, J=2.6, 1.5 Hz, 1H), 7.52 (dt, J=7.7, 1.2 Hz, 1H), 7.38 (t, J=7.9 Hz, 1H), 7.13-7.08 (m, 1H), 4.83 (s, 2H), 2.78 (q, J=7.6 Hz, 2H), 2.29 (s, 3H), 1.27 (t, J=7.6 Hz, 3H); ¹³C NMR (125 MHz, CDCl₃) (172.4, 164.0, 161.3, 159.7, 158.7, 147.7, 146.7, 137.7, 133.1, 130.5, 130.0, 130.0, 129.6, 128.5, 127.8, 119.9, 119.8, 118.7, 112.9, 109.0, 59.6, 19.3, 12.2, 10.2. LRMS (ESI): m/z 431.2 calc. for C₂₄H₂₃N₄O₄⁺ [M+H]⁺, found 431.7.

N′-(3-((3-methyl-5-propylisoxazol-4-yl)methoxy)benzoyl)quinoline-2-carbohydrazide (19, 78%): ¹H NMR (500 MHz, CDCl₃) δ 10.70 (s, 1H), 9.22 (s, 1H), 8.35 (d, J=8.5 Hz, 1H), 8.26 (d, J=8.4 Hz, 1H), 8.18 (d, J=8.5 Hz, 1H), 7.91 (dd, J=8.2, 1.4 Hz, 1H), 7.82 (ddd, J=8.4, 6.8, 1.4 Hz, 1H), 7.67 (ddd, J=8.1, 6.9, 1.2 Hz, 1H), 7.55 (dd, J=2.7, 1.5 Hz, 1H), 7.53-7.49 (m, 1H), 7.41 (t, J=7.9 Hz, 1H), 7.14 (dd, J=8.0, 2.6 Hz, 1H), 4.85 (s, 2H), 2.74 (t, J=7.5 Hz, 2H), 2.31 (s, 3H), 1.73 (h, J=7.4 Hz, 2H), 0.96 (t, J=7.4 Hz, 3H); ¹³C NMR (125 MHz, CDCl₃) δ 171.4, 163.9, 161.1, 159.7, 158.8, 147.7, 146.7, 137.7, 133.1, 130.5, 130.1, 130.0, 129.6, 128.5, 127.8, 119.8, 118.7, 113.0, 109.7, 59.6, 27.6, 21.2, 13.7, 10.2. MS (ESI): m/z 445.2 calc. for C₂₅H₂₅N₄O₄⁺ [M+H]⁺, found 445.7.

N′-(3-((3-methyl-5-phenylisoxazol-4-yl)methoxy)benzoyl)quinoline-2-carbohydrazide (20, 60%): ¹H NMR (500 MHz, CDCl₃) δ 10.67 (s, 1H), 9.43 (s, 1H), 8.31 (d, J=8.4 Hz, 1H), 8.22 (d, J=8.4 Hz, 1H), 8.16 (d, J=8.5 Hz, 1H), 7.89 (dd, J=8.2, 1.4 Hz, 1H), 7.80 (ddd, J=8.5, 6.9, 1.4 Hz, 1H), 7.72-7.63 (m, 3H), 7.58 (dd, J=2.6, 1.4 Hz, 1H), 7.53 (dt, J=7.7, 1.2 Hz, 1H), 7.46 (qd, J=4.8, 1.6 Hz, 3H), 7.40 (t, J=7.9 Hz, 1H), 7.15 (dd, J=8.2, 2.5 Hz, 1H), 4.99 (s, 2H), 2.38 (s, 3H); ¹³C NMR (125 MHz, CDCl₃) δ 168.4, 164.0, 161.3, 160.8, 158.6, 147.7, 146.6, 137.7, 133.2, 130.5, 130.4, 130.1, 130.0, 129.6, 129.1, 128.5, 127.8, 127.5, 127.4, 120.1, 119.7, 118.6, 113.1, 109.5, 59.9, 10.2. MS (ESI): m/z 479.2 calc. for C₂₈H₂₃N₄O₄⁺ [M+H]⁺, found 479.8.

N′-(3-((5-ethyl-3-methylisoxazol-4-yl)methoxy)benzoyl)quinoline-2-carbohydrazide (21): To a solution of 31 (200 mg, 0.77 mmol) in THF/H₂O (7.0 mL, 1:1 in volume) was added LiOH (36.7 mg, 1.53 mmol) at room temperature. The resulting mixture was stirred for 12 hours. Upon completion, the resultant mixture was acidified with aq. HCl then extracted with EtOAc. The organic extracts were combined, dried over Na₂SO₄, filtered, evaporated, and the residue was subjected to a quick flash column chromatography to afford the acid for the next step.

To a solution of the above acid (50 mg, 0.2 mmol) and catalytic amount of DMF in anhydrous CH₂Cl₂ (1 mL) at 0° C. was added (COCl)₂ (34 μL, 0.4 mmol) dropwise and the resulting mixture was stirred for 1 hour. The resulting mixture was concentrated under reduced pressure to afford the acid chloride 45 which was subjected to the next step without further purification.

To a solution of the acid chloride from the previous step in anhydrous CH₂Cl₂ at 0° C. was added DIPEA (42 μL, 0.25 mmol) dropwise followed by N-Boc-ethylenediamine (32 μL, 0.2 mmol). The resulting mixture was stirred at room temperature for 12 hours. The resulting mixture was concentrated under reduced pressure to afford the amide, which was subjected to the next step without further purification.

To a solution of the amide from the previous step in CH₂Cl₂ (0.6 mL) at 0° C. was added TFA (0.2 mL) dropwise. After 10 min, the resulting mixture was concentrated under reduced pressure to afford the primary amine 48, which was subjected to the next step without further purification.

Quinaldoyl chloride 37 (26 mg, 0.1 mmol) in CH₂Cl₂ (1.0 mL) was added dropwise to a dried round flask containing the primary amine 48 from the previous step, Et₃N (38 μL, 0.27 mmol), in CH₂Cl₂ (1.0 mL) at 0° C. The mixture was stirred at room temperature for 12 hours. The resulting mixture was concentrated under reduced pressure. The crude product was purified by flash column chromatography to obtain 21 (16.7 mg, 12% over 5 steps). ¹H NMR (500 MHz, CDCl₃) δ 8.73 (t, J=6.3 Hz, 1H), 8.32-8.25 (m, 2H), 8.10 (dq, J=8.6, 0.9 Hz, 1H), 7.90-7.87 (m, 1H), 7.77 (ddd, J=8.4, 6.9, 1.4 Hz, 1H), 7.63 (ddd, J=8.1, 6.9, 1.2 Hz, 1H), 7.58 (d, J=4.9 Hz, 1H), 7.52 (dd, J=2.6, 1.6 Hz, 1H), 7.44 (ddd, J=7.6, 1.6, 1.0 Hz, 1H), 7.35 (t, J=7.9 Hz, 1H), 7.04 (ddd, J=8.2, 2.6, 1.0 Hz, 1H), 4.83 (s, 2H), 3.84 (td, J=6.9, 6.2, 4.3 Hz, 2H), 3.77 (ddd, J=7.2, 4.8, 3.5 Hz, 2H), 2.41 (s, 3H), 2.28 (s, 3H); ¹³C NMR (125 MHz, CDCl₃) δ 167.6, 167.4, 166.6, 159.8, 158.5, 149.1, 146.5, 137.7, 135.8, 130.4, 129.8, 129.7, 129.4, 128.2, 127.8, 119.6, 118.9, 118.6, 112.8, 110.1, 59.6, 42.2, 39.3, 11.2, 10.2. HRMS (ESI): m/z 445.1870 calc. for C₂₅H₂₅N₄O₄⁺ [M+H]⁺, found 445.1876.

N′-(3-((3-methyl-5-propylisoxazol-4-yl)methoxy)benzoyl)quinoline-2-carbohydrazide (22): To a solution of acid chloride 45 (0.33 mmol) in anhydrous CH₂Cl₂ at 0° C. was added DIPEA (70 μL, 0.40 mmol) dropwise followed by N-Boc-1,3-propanediamine (58 μL, 0.33 mmol). The resulting mixture was stirred at room temperature for 12 hours. The resulting mixture was concentrated under reduced pressure to afford the amide, which was subjected to the next step without further purification.

To a solution of the amide from the previous step in CH₂Cl₂ (1.5 mL) at 0° C. was added TFA (0.5 mL) dropwise. After 10 min, the resulting mixture was concentrated under reduced pressure to afford the primary amine 49, which was subjected to the next step without further purification.

Quinaldoyl chloride 37 (63 mg, 0.33 mmol) in CH₂Cl₂ (1.5 mL) was added dropwise to a dried round flask containing the primary amine 49 from previous step, pyridine (0.13 mL, 1.66 mmol), and DMAP (4.0 mg, 0.03 mmol) in CH₂Cl₂ (1.5 mL) at 0° C. The mixture was stirred at room temperature for 12 hours. The mixture was then washed with dilute aqueous HCl and water and dried over Na₂SO₄. After removal of the solvent at reduced pressure, the crude product was purified by flash column chromatography to obtain 22 (50 mg, 23% over 5 steps) as product. ¹H NMR (500 MHz, CDCl₃) δ 8.56 (t, J=7.3 Hz, 1H), 8.37-8.27 (m, 2H), 8.12 (t, J=8.8 Hz, 1H), 7.91 (t, J=8.7 Hz, 1H), 7.78 (h, J=6.4, 4.1 Hz, 2H), 7.68-7.61 (m, 2H), 7.57 (t, J=8.4 Hz, 1H), 7.39 (q, J=8.4, 7.9 Hz, 1H), 7.28 (d, J=1.6 Hz, 1H), 7.10-7.05 (m, 1H), 4.89 (d, J=9.1 Hz, 2H), 3.71 (q, J=6.2 Hz, 2H), 3.56 (q, J=5.9 Hz, 2H), 2.44 (d, J=9.3 Hz, 3H), 2.31 (dd, J=8.1, 1.9 Hz, 3H), 1.95 (q, J=6.0 Hz, 2H); ¹³C NMR (125 MHz, CDCl₃) δ 167.6, 166.9, 165.8, 159.8, 158.6, 149.2, 146.5, 137.7, 136.2, 130.3, 129.8, 129.7, 129.4, 128.2, 127.8, 119.6, 118.8, 118.7, 112.9, 110.2, 59.6, 36.4, 36.1, 29.8, 11.2, 10.2. HRMS (ESI): m/z 481.1846 calc. for C₂₆H₂₆N₄NaO₄⁺ [M+Na]⁺, found 481.1851.

N′-(3-((3-methyl-5-phenylisoxazol-4-yl)methoxy)benzoyl)quinoline-2-carbohydrazide (23): 2-Quinolineethanamine 50 (70 mg, 0.41 mmol) in CH₂Cl₂ (2.0 mL) was added dropwise to a dried round flask containing the acid chloride 45 (0.41 mmol), pyridine (0.16 mL, 2.02 mmol), and DMAP (5.0 mg, 0.04 mmol) in CH₂Cl₂ (2.0 mL) at 0° C. The mixture was stirred at room temperature for 12 hours and then washed with dilute aqueous HCl and water and dried over Na₂SO₄. After removal of the solvent at reduced pressure, the crude product was purified by flash column chromatography to obtain 23 (0.8 mg; ˜5%). ¹H NMR (500 MHz, CDCl₃) δ 8.13 (dd, J=8.4, 0.9 Hz, 1H), 8.06-7.99 (m, 2H), 7.82 (dd, J=8.1, 1.4 Hz, 1H), 7.69 (ddd, J=8.4, 6.9, 1.4 Hz, 1H), 7.53 (ddd, J=8.1, 6.9, 1.2 Hz, 1H), 7.49 (dd, J=2.6, 1.5 Hz, 1H), 7.40-7.32 (m, 3H), 7.04 (ddd, J=7.8, 2.7, 1.3 Hz, 1H), 4.82 (s, 2H), 4.03-3.95 (m, 2H), 3.31-3.25 (m, 2H), 2.41 (s, 3H), 2.28 (s, 3H); ¹³C NMR (125 MHz, CDCl₃) δ 167.6, 166.7, 160.5, 159.8, 158.6, 147.6, 136.9, 136.5, 129.8, 129.7, 128.6, 127.8, 126.9, 126.3, 122.0, 119.3, 118.5, 113.0, 110.1, 59.6, 38.6, 37.0, 11.2, 10.2. HRMS (ESI): m/z 402.1812 calc. for C₂₄H₂₄N₃O₃⁺ [M+H]⁺, found 402.1817.

N-(3-((3,5-dimethylisoxazol-4-yl)methoxy)benzoyl)quinoline-2-carboxamide (24): Concentrated ammonia (4.0 mL) was added dropwise to a solution of 31 (300 mg, 1.14 mmol) in MeOH (2.0 mL). The reaction mixture was heated to 60° C. and stirred for 12 hours until completion, as determined by TLC. After cooling, water was added, and the precipitate was filtered and washed with a small amount of methanol and water. The crude product was put under vacuum to afford amide (150 mg, 53%) as product, which was subjected to the next step without further purification.

To a solution of the above amide (20 mg, 0.08 mmol) in anhydrous THF was added NaH (6.4 mg, 60% in mineral oil) at 0° C. The resulting mixture was stirred at 0° C. for 30 min before adding 37 (15.3 mg, 0.08 mmol). The mixture was stirred at room temperature for 12 hours and then diluted with EtOAc. The organic phase was washed with dilute aqueous HCl and water and dried over Na₂SO₄. After removal of the solvent at reduced pressure, the crude product was purified by flash column chromatography to obtain 24 (15 mg, 47%). ¹H NMR (500 MHz, CDCl₃) δ 11.77 (s, 1H), 8.42 (d, J=1.3 Hz, 2H), 8.19 (dd, J=8.6, 1.1 Hz, 1H), 7.96 (dd, J=8.2, 1.4 Hz, 1H), 7.85 (ddd, J=8.5, 6.9, 1.4 Hz, 1H), 7.74-7.63 (m, 3H), 7.52 (t, J=7.9 Hz, 1H), 7.22 (ddd, J=8.2, 2.7, 0.9 Hz, 1H), 4.92 (s, 2H), 2.47 (s, 3H), 2.33 (s, 3H); ¹³C NMR (125 MHz, CDCl₃) δ 167.8, 164.6, 162.2, 159.8, 158.9, 148.3, 146.2, 138.4, 135.1, 130.8, 130.2, 130.0, 129.9, 129.0, 128.0, 120.6, 119.9, 119.0, 113.8, 109.9, 59.8, 11.3, 10.2. MS (ESI): m/z 402.1 calc. for C₂₃H₂₀N₃O₄⁺ [M+H]⁺, found 402.6.

N-(3-((3,5-dimethylisoxazol-4-yl)methoxy)benzyl)quinoline-2-carboxamide (25): To a solution of 31 (390 mg, 1.49 mmol) in anhydrous THF (2 mL) at 0° C. was slowly added LiAlH₄ (34 mg, 0.90 mmol) in anhydrous THF (2 mL) dropwise. The resulting mixture was stirred at room temperature for 3 hours. The reaction was quenched by subsequent addition of water and EtOAc. The suspension was filtered, and the residue was extracted with EtOAc, then dried over Na₂SO₄. After removal of the solvent at reduced pressure, the crude residue was purified by flash column chromatography to afford the primary alcohol (340 mL, 98% yield). ¹H NMR (500 MHz, CDCl₃) δ 7.31-7.25 (m, 1H), 7.00-6.96 (m, 2H), 6.86 (ddd, J=8.2, 2.6, 1.0 Hz, 1H), 4.80 (s, 2H), 4.69 (s, 2H), 2.40 (s, 3H), 2.28 (s, 3H); ¹³C NMR (125 MHz, CDCl₃) δ 167.5, 159.8, 158.6, 142.8, 129.8, 119.8, 114.1, 113.0, 110.3, 65.0, 59.5, 11.2, 10.2.

To a solution of the above primary alcohol (340 mg. 1.46 mmol) in anhydrous CH₂Cl₂ (14.6 mL) at 0° C. was added Dess-Martin periodinane (680 mg, 1.60 mmol). The mixture was stirred at room temperature for 3 hours before water was added. The resultant mixture was extracted with CH₂Cl₂, washed with aq. Na₂S2O₃ and aq. NaHCO₃. The organic extracts were combined, dried over Na₂SO₄, filtered, and evaporated. The crude residue was purified by flash column chromatography to afford aldehyde 51 (297 mg, 880% yield). ¹H NMR (500 MHz, CDCl₃) δ 9.96 (s, 1H), 7.53-7.39 (m, 3H), 7.19 (ddd, J=7.6, 2.7, 1.7 Hz, 1H), 4.85 (s, 2H), 2.40 (s, 3H), 2.27 (s, 3H); ¹³C NMR (125 MHz, CDCl₃) δ 191.9, 167.7, 159.7, 158.9, 137.9, 130.3, 124.4, 122.3, 112.4, 109.9, 59.7, 11.2, 10.2.

To a solution of 51 in MeOH/H₂O (2 mL, 1:1 in volume) was added H₂NOH—HCl (36 mg, 0.52 mmol) and NaOH (21 mg, 0.52 mmol). The mixture was refluxed for 6 hours. The resultant mixture was extracted with EtOAc. The organic extracts were combined, dried over Na₂SO₄, filtered, and evaporated. The crude was subjected to the next step without further purification.

To a solution of the oxime from the previous step in anhydrous THF (1 mL) at 0° C. was slowly added LiAl—H₄ (36 mg, 0.95 mmol) in anhydrous THF (1 mL) dropwise. The resulting mixture was raised to room temperature and then stirred at reflux for 3 hours. The reaction was quenched by subsequent addition of water and EtOAc. The suspension was filtered, and the residue was extracted with EtOAc, then dried over Na₂SO₄. After removal of the solvent at reduced pressure, the crude was subjected to the next step without further purification.

Quinaldoyl chloride 37 (16.5 mg, 0.086 mmol) in CH₂Cl₂ (1.0 mL) was added dropwise to a dried round flask containing the primary amine from the previous step, DIPEA (18 μL, 0.103 mmol in CH₂Cl₂ (1.0 mL) at 0° C. The mixture was stirred at room temperature for 12 hours. After removal of the solvent at reduced pressure, the crude product was purified by flash column chromatography to obtain 25 (10 mg, 6% yield, over 3 steps) as product. ¹H NMR (500 MHz, CDCl₃) δ 8.63 (s, 1H), 8.37-8.31 (m, 2H), 8.08 (dd, J=8.6, 1.1 Hz, 1H), 7.89 (dt, J=8.1, 1.0 Hz, 1H), 7.76 (ddd, J=8.4, 6.8, 1.5 Hz, 1H), 7.63 (ddd, J=8.1, 6.9, 1.2 Hz, 1H), 7.31 (t, J=7.9 Hz, 1H), 7.08-7.04 (m, 1H), 7.01 (t, J=2.1 Hz, 1H), 6.90-6.86 (m, 1H), 4.79 (s, 2H), 4.74 (d, J=6.2 Hz, 2H), 2.38 (s, 3H), 2.27 (s, 3H); ¹³C NMR (125 MHz, CDCl₃) δ 167.5, 164.5, 159.8, 158.7, 149.6, 146.5, 140.2, 137.6, 130.2, 129.9, 129.7, 129.4, 128.0, 127.8, 120.9, 119.0, 114.1, 113.9, 110.2, 59.4, 43.5, 11.2, 10.2. HRMS (ESI): m/z 410.1475 calc. for C₂₃H₂₁N₃NaO₃⁺ [M+Na]⁺, found 410.1482.

N-(3-((3,5-dimethylisoxazol-4-yl)methoxy)phenethyl)quinoline-2-carboxamide (26): To a solution of 53 (428 mg, 1.58 mmol) in anhydrous THF (2.5 mL) at 0° C. was slowly added LiAlH₄ (36 mg, 0.95 mmol) in anhydrous THF (2.5 mL) dropwise. The resulting mixture was stirred at room temperature for 3 hours. The reaction was quenched by subsequent addition of water and EtOAc. The suspension was filtered, and the residue was extracted with EtOAc, then dried over Na₂SO₄. After removal of the solvent at reduced pressure, the crude residue was purified by flash column chromatography to afford the primary alcohol (300 mg, 78% yield).

¹H NMR (500 MHz, CDCl₃) δ 7.30-7.19 (m, 1H), 6.90-6.85 (m, 1H), 6.82 (dd, J=7.5, 1.1 Hz, 2H), 4.78 (s, 2H), 3.85 (t, J=6.6 Hz, 2H), 2.85 (t, J=6.6 Hz, 2H), 2.39 (s, 3H), 2.28 (s, 3H); ¹³C NMR (125 MHz, CDCl₃) δ 167.6, 159.8, 158.6, 140.5, 129.7, 122.1, 115.7, 112.6, 110.4, 63.5, 59.4, 39.2, 11.2, 10.2.

To a solution of the above primary alcohol (300 nig 1.21 mmol) in anhydrous CH₂Cl₂ (7 mL) at 0° C. was added Dess-Martin periodinane (618 rug, 1.46 mmol). The mixture was stirred at room temperature for 3 hours. Water was added. The resultant mixture was extracted with CH₂Cl₂, washed with aq. Na₂S₂O₃ and aq. NaHCO₃. The organic extracts were combined, dried over Na₂SO₄, filtered, evaporated, and the crude residue was purified by flash column chromatography to afford the aldehyde (230 mg, 78% yield). ¹H NMR (500 MHz, CDCl₃) δ 9.73 (s, 1H), 7.29 (dd, J=8.3, 7.5 Hz, 1H), 6.91-6.82 (m, 2H), 6.79 (dd, J=2.6, 1.6 Hz, 1H), 4.78 (s, 2H), 3.66 (d, J=2.4 Hz, 2H), 2.38 (s, 3H), 2.27 (s, 3H); ¹³C NMR (125 MHz, CDCl₃) δ 199.1, 167.6, 159.8, 158.8, 133.6, 130.2, 122.7, 116.2, 113.7, 110.2, 59.5, 50.5, 11.1, 10.1.

To a solution of the above aldehyde (125 mg, 0.51 mmol) in H)O (1 mL) was added H₂NOH—HCl (46.7 mg, 0.66 mmol) and Na₂CO₃ (32.4 mg, 0.31 mmol). The mixture was refluxed for 6 hours. The resultant mixture was extracted with EtOAc. The organic extracts were combined, dried over Na₂SO₄, filtered, and evaporated. The crude was subjected to the next step without further purification.

To a solution of the oxime from the previous step in anhydrous THF (1.5 mL) at 0° C. was slowly added LiAlH₄ (28 mg, 0.75 mmol) in anhydrous THF (1.5 mL) dropwise. The resulting mixture was raised to room temperature for 3 hours. The reaction was quenched by subsequent addition of water and EtOAc. The suspension was filtered, and the residue was extracted with EtOAc, then dried over Na₂SO₄. After removal of the solvent at reduced pressure, the crude was subjected to the next step without further purification.

Quinaldoyl chloride 37 (27 mg, 0.14 mmol) in CH₂Cl₂ (1.4 mL) was added dropwise to a dried round flask containing the primary amine 54 from the previous step (35 mg), DIPEA (28 μL, 0.16 mmol in CH₂Cl₂ (1.4 mL) at 0° C. The mixture was stirred at room temperature for 12 hours. After removal of the solvent at reduced pressure, the crude product was purified by flash column chromatography to obtain 22 (7.4 mg, 10% yield, over 3 steps) as product. ¹H NMR (500 MHz, CDCl₃) δ 8.40 (s, 1H), 8.31 (s, 2H), 8.07-8.03 (m, 1H), 7.88 (dd, J=8.3, 1.5 Hz, 1H), 7.74 (ddd, J=8.4, 6.8, 1.4 Hz, 1H), 7.62 (ddd, J=8.1, 6.9, 1.2 Hz, 1H), 7.28 (t, J=7.9 Hz, 1H), 6.95 (dt, J=7.6, 1.2 Hz, 1H), 6.90 (t, J=2.1 Hz, 1H), 6.84 (ddd, J=8.2, 2.7, 1.0 Hz, 1H), 4.76 (s, 2H), 3.80 (dt, J=7.5, 6.5 Hz, 2H), 3.00 (t, J=7.2 Hz, 2H), 2.35 (s, 3H), 2.24 (s, 3H); ¹³C NMR (125 MHz, CDCl₃) δ 167.5, 164.5, 159.8, 158.7, 149.8, 146.5, 140.9, 137.5, 130.1, 129.8, 129.6, 129.3, 128.0, 127.8, 122.0, 118.8, 115.3, 113.0, 110.3, 59.5, 40.8, 36.1, 11.1, 10.1. HRMS (ESI): m/z 424.1632 calc. for C₂₄H₂₃N₃NaO₃⁺ [M+Na]⁺, found 424.1642.

What is claimed is:

1. A compound of the formula 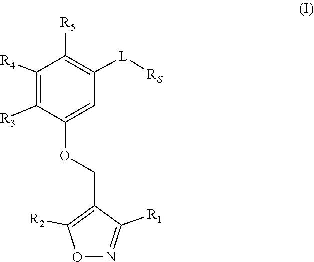
or a salt, hydrate, or solvate thereof;
wherein
R₁ and R₂ are independently selected from the list consisting of alkyl, aryl, arylalkyl, alkenyl, cycloalkyl, heteroalkyl, heteroaryl, heteroarylalkyl, heteroalkenyl, and heterocycloalkyl, each of which is optionally substituted;
R₃, R₄ and R₅ are each independently hydrogen or —(CH₂)ₓZ^{X}, where x is an integer from 0-6 and Z^{X} is halogen, hydroxy, C₁-C₆ alkanoyloxy, optionally substituted aroyloxy, C₁-C₆ alkyl, C₁-C₆ alkoxy, C₃-C₈ cycloalkyl, C₃-C₈ cycloalkoxy, C₂-C₆ alkenyl, C₂-C₆ alkynyl, C₁-C₆ haloalkyl, C₁-C₆ haloalkoxy, C₃-C₈ halocycloalkyl, C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, nitro; —CO₂R₆, or —CONR₇R₈, where R₆, R₇, and R₈ are each independently selected in each instance from hydrogen, C₁-C₆ alkyl, aryl-C₁-C₆ alkyl, and heteroaryl-C₁-C₆ alkyl or R₇, R₈, and the nitrogen to which they are attached form an optionally substituted heterocycle;
L is a linker;
R_{S} is cycloalkyl, aryl or heteroaryl, each of which is optionally substituted; or R_{S} is —NR₉R₁₀, where R₉, and R₁₀ are each independently selected in each instance from hydrogen, C₁-C₆ alkyl, aryl-C₁-C₆ alkyl, and heteroaryl-C₁-C₆ alkyl; or R₉, R₁₀, and the nitrogen to which they are attached form an optionally substituted heterocycle;
with the proviso that the compound is not

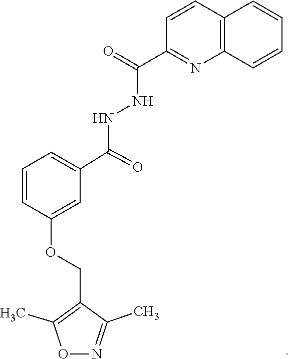 [SMILES: Cc1noc(C)c1COc1cccc(C(=O)NNC(=O)c2ccc3ccccc3n2)c1]

2. The compound, or a salt, a hydrate, or a solvate thereof, of claim 1, wherein L is C(O)NHNHC(O).

3. The compound, or a salt, a hydrate, or a solvate thereof, of claim 2, wherein R_{S} is quinolinyl.

4. The compound, or a salt, a hydrate, or a solvate thereof, of claim 3, wherein each of R₃, R₄ and R₅ is a hydrogen, and each of R₁ and R₂ is an alkyl.

5. The compound, or a salt, a hydrate, or a solvate thereof of claim 1, wherein L is selected from the group consisting of C(O)(NH)_{N}C(O), where N is 1 or 2; C(O)NHNHSO₂, C(O)NH(CH₂)_{M}NHC(O), where M is an integer from 1 to about 6; C(O)NH(CH₂)_{M2}NHSO₂, where M2 is an integer from 1 to about 6; C(O)NH(CH₂)_{M3}, where M3 is an integer from 1 to about 6, and SO₂NH(CH₂)_{M4}, where M4 is an integer from 1 to about 6; HNC(O)(CH₂)_{M4}C(O)NH, were M4 is an integer from 0 to about 4; and 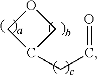
where a and b are independently 0, 1, or 2, with the proviso that a and b cannot both be 0, and c is an integer from 1 to about 4.

6. A method of inhibiting protein arginine methyltransferase 5 (PRMT5) in a patient in need thereof, the method comprising the step of administering to the patient one or more compounds of the formula 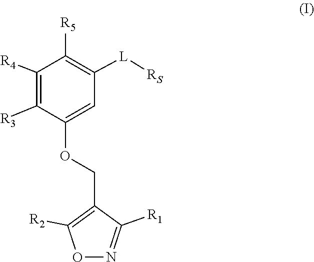
or salts, hydrates, or solvates thereof,
wherein
R₁ and R₂ are independently selected from the list consisting of alkyl, aryl, arylalkyl, alkenyl, cycloalkyl, heteroalkyl, heteroaryl, heteroarylalkyl, heteroalkenyl, and heterocycloalkyl, each of which is optionally substituted;
R₃, R₄ and R₅ are each independently hydrogen or —(CH₂)ₓZ^{X}, where x is an integer from 0-6 and Z^{X} is halogen, hydroxy, C₁-C₆ alkanoyloxy, optionally substituted aroyloxy, C₁-C₆ alkyl, C₁-C₆ alkoxy, C₃-C₈ cycloalkyl, C₃-C₈ cycloalkoxy, C₂-C₆ alkenyl, C₂-C₆ alkynyl, C₁-C₆ haloalkyl, C₁-C₆ haloalkoxy, C₃-C₈ halocycloalkyl, C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, nitro; —CO₂R₆, or —CONR₇R₈, where R₆, R₇, and R₈ are each independently selected in each instance from hydrogen, C₁-C₆ alkyl, aryl-C₁-C₆ alkyl, and heteroaryl-C₁-C₆ alkyl or R₇, R₈, and the nitrogen to which they are attached form an optionally substituted heterocycle;
L is a linker;
R_{S} is cycloalkyl, aryl or heteroaryl, each of which is optionally substituted; or R_{S} is —NR₉R₁₀, where R₉, and R₁₀ are each independently selected in each instance from hydrogen, C₁-C₆ alkyl, aryl-C₁-C₆ alkyl, and heteroaryl-C₁-C₆ alkyl; or R₉, R₁₀, and the nitrogen to which they are attached form an optionally substituted heterocycle.

7. The method of claim 6, wherein L is C(O)NHNHC(O).

8. The method of claim 7, wherein R_{S} is quinolinyl.

9. The method of claim 8, wherein each of R₃, R₄ and R₅ is a hydrogen, and each of R₁ and R₂ is an alkyl.

10. The method of claim 6, wherein L is selected from the group consisting of C(O)(NH)_{N}C(O), where N is 1 or 2; C(O)NHNHSO₂, C(O)NH(CH₂)_{M}NHC(O), where M is an integer from 1 to about 6; C(O)NH(CH₂)_{M2}NHSO₂, where M2 is an integer from 1 to about 6; C(O)NH(CH₂)_{M3}, where M3 is an integer from 1 to about 6, and SO₂NH(CH₂)_{M4}, where M4 is an integer from 1 to about 6; HNC(O)(CH₂)_{M4}C(O)NH, were M4 is an integer from 0 to about 4; and 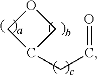
where a and b are independently 0, 1, or 2, with the proviso that a and b cannot both be 0, and c is an integer from 1 to about 4.

11. A pharmaceutical composition comprising a therapeutically effective amount of one or more compounds or salts, hydrates, or solvates described in any of claims 1-5, and at least one pharmaceutically acceptable carrier or excipient.

12. A method of inhibiting protein arginine methyltransferase 5 (PRMT5) in a patient in need thereof, the method comprising the step of administering to the patient the composition of claim 11.
